(19) United States
(12) Patent Application Publication
(10) Pub. No.: US 2024/0240126 A1
(43) Pub. Date: Jul. 18, 2024

(54) Microfluidic Barcode-Like Cell Sensor For Microscope-Free Quantitative Detection Of Small Amount Of Cells

(71) Applicant: Hong Kong Baptist University, Hong Kong (CN)

(72) Inventors: Kangning REN, Hong Kong (CN); Xiaowen CHU, Hong Kong (CN); Chiu Wing CHAN, Hong Kong (CN); Han SUN, Hong Kong (CN); Yisu WANG, Hong Kong (CN); Zhihao ZHAO, Hong Kong (CN); Sin Yung SIU, Hong Kong (CN)

(21) Appl. No.: 18/561,844

(22) PCT Filed: May 24, 2022
(86) PCT No.: PCT/CN2022/094665

§ 371 Date: Nov. 17, 2023

Related U.S. Application Data
(60) Provisional application No. 63/192,559, filed on May 24, 2021.

(51) Int. Cl. C12M 3/06; C12M 1/00; C12M 1/34; C12Q 1/18

(52) U.S. Cl. CPC C12M 23/16; C12M 29/04; C12M 41/36; C12Q 1/18

(57) ABSTRACT

Provided herein is a resource-independent and cost-efficient antimicrobial susceptibility testing (AST) system or apparatus that can rapidly process a large number of samples. The AST system includes a barcode-like cell sensor based on an adaptive linear filter array for implementing a fully automatic and microscope-free method for counting a very small volume of cells in samples, wherein suspended cells concentrate into microbars with various lengths proportional to the number of cells. The AST system also includes an on-chip culture that takes much less time than standard methods, thereby realizing a low-cost and resource-independent platform for portable AST, from which results can be obtained through a portable device such as a cell phone.

### CROSS-REFERENCE TO RELATED APPLICATION

This application claims priority from a U.S. provisional patent application No. 63/192,559 filed on May 24, 2021, which disclosure is incorporated herein by reference.

### FIELD OF THE INVENTION

The present invention relates to a cell sensor for counting very small volumes of cells based on an adaptive linear filter array. More particularly, it relates to a method of using a fully automatic and microscope-free cell sensor for counting very small volumes of cells, where suspended cells concentrate into microbars with various lengths proportional to the number of cells present in a sample.

### BACKGROUND OF THE INVENTION

In the past few decades, antimicrobials have been treated as the panacea of bacterial infections. However, the widespread overuse and misuse of antimicrobials by the public, physicians, and agricultural industries has accelerated the natural development of antimicrobial resistance (AMR) in certain microorganisms. The overuse/misuse of antibiotics is especially rampant. According to a recent high-profile report published in PNAS, the defined daily doses (DDD) of antibiotic consumption increased by 65% (from 21.1 to 34.8 billion DDDs) between 2000 and 2015. There is cause for concern, as AMR is one of the biggest threats to global health and modern civilization, and unless a global response is mounted, by 2050, an estimate of 10 million people a year will die due to it.

Clinically-related AMR has received the most focus from both the public and researchers, and many novel antimicrobial susceptibility testing (AST) methods are being developed to help physicians provide more personalized therapies. However, there has yet been sufficient tool to deal with AMR issues outside well-equipped healthcare facilities. These issues actually cover a wide range of situations, e.g., frequent surveys for the safety of water, food, and public facilities; an urgent survey of massive samples during a pandemic; or any AMR test in low-income countries. Taking the AMR in the environment as an example, reports proved that even a very low concentration of antibiotic-resistant bacteria in the environment can ultimately affect human health, not to mention the severe overuse of antimicrobials in the livestock industry of less regulated areas of the world. Meanwhile, the transportation of AMR bacteria from one place to another through various ways largely expands the range of AMR threat. Unfortunately, the lack of screening tools for AMR bacteria made us powerless in such situations. For example, reports have found a remarkable amount of antibiotic-resistant bacteria within environmental compartments worldwide, especially for those remote/underdeveloped areas, some with exceptionally high concentrations due to the pollution by various anthropogenic activities. To monitor such AMR risk in the mentioned situations, an efficient and resource-independent AST method is urgently needed, one that can rapidly screen the massive number of samples without the need of advanced clinical assay facilities; if samples suspected to contain AMR bacteria are detected in such a screening, they could then be sent back to the laboratory for conventional testing. In comparison to existing routine AST methods, such a potential AST tool would fill the gap between the facility-dependent standard tests and the need for massive AMR testing on-site or at the places where such facility resource is unavailable.

Current AST methods can be separated into two categories: phenotypic-based ASTs and genotypic-based ASTs. Phenotypic-based ASTs are frequently used as standard methods in real practice, which include the disk diffusion, broth microdilution, and E-test methods. These conventional methods are well-established, considered reliable, and routinely performed in laboratories because they are culture-based methodologies that show the bacteriostatic effect of drugs on bacteria proliferation.

These methods determine the minimum inhibitory concentration (MIC) of antibiotic needed through observable cell number changes at the macroscopic scale, which typically requires at least 9-20 hours of incubation time, which is unsuitable for the routine screening of the massive samples on-site. Actually, additional pre-incubation time is often needed when the initial bacteria density is not high in the sample. For instance, MIC value determination via the broth dilution method typically requires 105 cells. Moreover, these methods are usually labor-intensive, time-consuming, and expensive to operate, which is a significant drawback for on-site mass-screening. When considering extreme cases like the outbreak of a pandemic, the fatal weakness for the culture-based phenotypic ASTs is only exacerbated when a large number of time-sensitive suspected samples needs to be processed at the same time.

To realize rapid AST, researchers have proposed various genotypic-based methods, such as whole-genome sequencing and polymerase chain reaction (PCR)-based methods. However, such methods are still impractical for use under resource-limited conditions (e.g., the evaluation of AMR in remote/underdeveloped areas or an urgent situation which requires to perform massive tests etc.) due to high technical complexities and high equipment/consumable costs. Also, PCR-based AST methods require detailed prior knowledge of the bacterial gene(s) encoding for resistance in order to design the necessary primer(s), so if new, unknown resistance mechanisms have arisen, false-negative results may occur. On the other hand, false-positive results may arise if a gene with known AMR function in a sample is detected.

It is undoubted that both rapidness and reliability are essential to AMR testing; to this end, microfluidic-based phenotypic ASTs have been investigated by many researchers in the past decade. Microfluidic AST approaches can reduce the time needed to detect AMR and determine MIC values to just 2-3 hours by observing bacteria at the single-cell level. For example, in the present inventors' previous paper, a polypropylene (PP) microfluidic chip utilizing a single-cell strategy was realized for rapid and low-cost AST, wherein the MIC could be assessed by viewing cell morphology and surface chemical changes under a microscope. In addition, microfluidic ASTs require a much smaller amount of sample than conventional ASTs. However, such existing devices rely upon expensive and cumbersome instrumentation for analysis, e.g., a high-quality microscope for inspecting individual cells, which hampers the AST device's portability and accessibility for quick and high-throughput on-site screening for AMR bacteria under resource-limited conditions.

In summary, most of the AST methods currently used for the assessment of potential antimicrobial resistant environmental samples are still based on the culture-based and genotypic-based methodologies. Most of these methods still suffer from labor-intensive, expensive, and time-consuming issues, rendering them unsuitable for use in a large-scale and routine testing requiring point-of-care-testing (POCT) in resource-limited conditions. Although some advanced ASTs have been developed, they are still hampered by various limitations, such as high operation cost, high technical background requirements, and reliance on sophisticated and expensive instruments. These limitations render current ASTs unsuitable for practical implementation in remote and resource-limited areas. Some POCT devices have been developed to resolve the challenges faced in these situations, aiming to realize AST under a resource-limited condition. However, most of them are still impeded from different limitations, such as poor performance in quantitative aspect, limited universality, and even a long turnaround time, making them incapable of providing rapid AST function, i.e., obtaining quantitative MIC values timely. In general, the lack of a microscope-free method that can analyze very small amounts of bacteria has been a major hurdle to enable rapid culture-based AST in resource-limited conditions (FIG. 1). For the conventional and the automated system-based ASTs which are well-established and culture-based methodologies, 16-24 hours and 6.5-12 hours are typically required for reporting AST results. In comparison, the ASTs using a single-cell strategy will have an earlier point for MIC detection, which only takes 2-3 hours for reporting AST results. However, those reported microfluidic-based ASTs still suffer from different limitations, e.g., high operation cost, reliance on sophisticated and expensive instruments, etc., which render them unsuitable for practical implementation in remote and resource-limited areas.

### SUMMARY OF THE INVENTION

Accordingly, it is an objective of the present invention to provide an accelerated phenotypic AST method without using single-cell inspection.

In one aspect of the present invention, there is provided a microfluidic-based platform for implementing a one-pot antimicrobial susceptibility testing (AST) including cell culture, drug-cell incubation, and microscope-free quantitative analysis of viable cells after treatment by one or more compounds, the microfluidic-based platform comprises: a thermal stage; a light source; and a visual inspection window, where the thermal stage includes a cell culture zone and a cell sensing zone, and where the cell culture zone includes at least three fluid inlets and a drug concentration gradient generator downstream with respect to the at least three fluid inlets, and where the drug concentration gradient generator includes a plurality of diverging fluid channels diverging fluids downstream with respect to at least two of the fluid inlets, and a plurality of micro-chambers each with one or more deepened microwells for cell culture;

the cell sensing zone includes a plurality of adaptive linear filter channels connecting to multiple fluid outlets of the cell culture zone via a one-piece connector, where each of the adaptive linear filter channels is configured to receive cultivated cells from the cell culture zone and subsequently the received cultivated cells will accumulate at a downstream end of each of the adaptive linear filter channels, the light source is disposed adjacent to or proximal to the downstream end of the adaptive linear filter channels for illuminating a wavelength of light towards the accumulated cultivated cells at the downstream end of each of the adaptive linear filter channels in order to visualize the accumulated cultivated cells through the visual inspection window disposed above an enclosure of the plurality of adaptive linear filter channels and image signal derived thereof being able to be directly captured by a portable device equipped with an imaging function.

In certain embodiments, each of the adaptive linear filter channels includes at least one main channel and at least two side channels disposed in a parallel orientation with the at least one main channel, and the at least one main channel communicates with the at least two side channels through a plurality of nano-scale channelization channels disposed in an orthogonal orientation with respect to both the at least one main channel and the at least two side channels such that the fluid containing cultivated cells flowing through the at least one main channel of the adaptive linear filter channel are directed to the at least two side channels through the plurality of nano-scale channelization channels based on filtration effect.

In certain embodiments, the at least one main channel and the at least two side channels have an identical cross-sectional area with an aspect ratio of channel height to channel width of less than 1.

In certain embodiments, the at least one main channel and the at least two side channels have an average channel height of about 8 μm.

In certain embodiments, the at least one main channel and the at least two side channels have an average channel width of about 16 μm.

In certain embodiments, the nano-scale channelization channels have the same channel width as that of the at least one main channel and the at least two side channels and an average channel height of about 800 nm.

In certain embodiments, the cell sensing zone comprises at least eight adaptive linear filter channels to form an array of adaptive linear filter channels with various lengths of visible microbars corresponding to various quantities of cells accumulated at each of the adaptive linear filter channels after treatment with the one or more compounds at different concentrations according to different fluid channels of the diverging fluid channels of the drug concentration gradient generator at the cell culture zone.

In certain embodiments, the length of the visible microbar is proportional to a proliferation rate of viable cells to be accumulated at the corresponding adaptive linear filter channel after the cells being treated with the one or more compounds at a specific concentration from one of the diverging fluid channels of the drug concentration gradient generator at the cell culture zone.

In certain embodiments, a drug-containing fluid is loaded into one of the fluid inlets disposed upstream with respect to the drug concentration gradient generator while a pure fluid is loaded into another fluid inlet also disposed upstream with respect to the drug concentration gradient generator.

In certain embodiments, the fluid containing cells is loaded into the fluid inlet disposed upstream with respect to the micro-chambers.

In certain embodiments, a sample containing bacteria of interest is initially injected into the cell culture zone through one of the fluid inlets; subsequently, antibiotic-doped media and media alone are injected into the cell culture zone via another two of the fluid inlets, respectively. The bacteria are cultured in the drug concentration gradient for about 1-3 hours and then pushed into the cell counting zone by increasing flow rate. Bacteria will then accumulate inside the adaptive linear filters in the barcode-like cell sensing zone, and after gram staining the results can be captured and analyzed by using a cell phone equipped with a macro lens adapter.

In certain embodiments, the increased flow rate of the fluid to push the bacterial culture from the cell culture zone to the barcode-like cell sensing zone is from about 0.15 μl/min to about 2 μl/min.

In other embodiments, the flow rate of the fluid to push the bacterial culture from the cell culture zone to the barcode-like cell sensing zone varies depending on multiple factors including, but not limited to, bacterial adherence, bacterial hydrophobicity, and surface adhesiveness or hydrophobicity of polymeric surface of the microfluidic channels inside the cell culture zone and cell sensing zone with respect to the bacteria of interest.

In certain embodiments, the faster the flow rate of the fluid is, the higher is the accumulation rate of the bacteria in the adaptive linear filters of the cell sensing zone.

In certain embodiments, the flow rate of the fluid is increased to a level that substantially no debonding of the channelization channels is observed.

In certain embodiments, the accumulated cells at the adaptive linear filter channels are gram stained such that under the illumination by the light source the image signal derived from the gram stained cells is directly captured by the portable device equipped with the imaging function, and wherein the wavelength of light illuminated by the light source is within a visible light range.

In certain embodiments, at least the cell culture zone and the cell sensing zone of the thermal stage are made of one or more thermoplastic materials that are biocompatible whilst the cells cultivated at the cell culture zone do not adhere on interior surface of the microfluidic channels of the cell culture zone when a pure fluid is loaded into one of the fluid inlets to flush the cultivated cells towards the downstream direction to the fluid outlets of the cell culture zone.

In certain embodiments, the thermoplastic materials include polypropylene.

In another aspect of the present invention, there is provided a system for screening or evaluating an antimicrobial activity of a potential drug candidate against one or more microbes, which includes an enclosure housing the microfluidic-based platform described herein, a fully automated fluid pump, a main circuit board and multiple control components for controlling loading of different fluids to the fluid inlets of the platform, a reagent compartment connecting to one or more fluid inlets of the microfluidic-based platform, a drug-cell incubation compartment with a thermostat for controlling temperature of the cell culture zone where the one or more microbes are incubated with different concentrations of the potential drug candidate, and a shadowless light panel for capturing images of the visualized accumulated cells at the cell sensing zone of the microfluidic-based platform from the visible inspection window by a macroscopic lens equipped to a portable device.

In other aspect of the present invention, there is provided a method for screening or evaluating an antimicrobial activity of a potential drug candidate against one or more microbes of interest, which includes:

loading the potential drug candidate into the reagent compartment of the system described herein;

using a corresponding software program installed in a paired workstation or portable device to control power on/off of and flowrate of fluid generated by the automated fluid pump of the system;

loading the one or more microbes of interest to the corresponding fluid inlet of the microfluidic-based platform;

treating the one or more microbes with the potential drug candidate at different concentrations in the cell culture zone of the microfluidic-based platform under a constant temperature controlled by the thermostat for a sufficient time duration in the drug-cell incubation compartment of the system;

flushing the cell culture of the one or more microbes after treating with different concentrations of the potential drug candidate from the cell culture zone via the one-piece connector to the cell sensing zone of the microfluidic-based platform by activating an antimicrobial susceptibility test function of the system through the software program of the paired workstation or portable device;

capturing images of visible bars derived from the accumulated cultivated cells of the one or more microbes at the adaptive linear filter array of the cell sensing zone by an imaging module paired with the workstation or the portable device under an illumination by the shadowless light panel of the system; converting the captured images into image data for various lengths of the visible bars;

comparing the image data for various lengths of the visible bars with a referenced image data to determine the antimicrobial activity of the potential drug candidate against the one or more microbes of interest.

In certain embodiments, the treating of the one or more microbes with the potential drug candidate at different concentrations in the cell culture zone of the microfluidic-based platform under a constant temperature controlled by the thermostat is for about 1 to 3 hours.

In certain embodiments, the one or more microbes comprise antimicrobial resistant (AMR) microbes.

In a further aspect of the present invention, there is provided a method for evaluating antimicrobial resistance of a microbe against a compound, which includes:

loading the compound into the reagent compartment of the system described herein;

using a corresponding software program installed in a paired workstation or portable device to control power on/off of and flowrate of fluid generated by the automated fluid pump of the system;

loading microbe to the corresponding fluid inlet of the microfluidic-based platform;

treating the microbe with the compound at different concentrations in the cell culture zone of the microfluidic-based platform under a constant temperature controlled by the thermostat for a sufficient time duration in the drug-cell incubation compartment of the system;

flushing the cell culture of the microbe after treating with different concentrations of the compound from the cell culture zone via the one-piece connector to the cell sensing zone of the microfluidic-based platform by activating an antimicrobial susceptibility test function of the system through the software program of the paired workstation or portable device;

capturing images of visible bars derived from the accumulated cultivated cells of the microbe at the adaptive linear filter array of the cell sensing zone by an imaging module paired with the workstation or the portable device under an illumination by the shadowless light panel of the system; converting the captured images into image data for various lengths of the visible bars; comparing the image data for various lengths of the visible bars with a referenced image data to determine the antimicrobial resistance of the microbe against the compound.

In certain embodiments, the treating of the microbe with the compound at different concentrations in the cell culture zone of the microfluidic-based platform under a constant temperature controlled by the thermostat is for about 1 to 3 hours.

In certain embodiments, the compound used in evaluating antimicrobial resistance of a microbe is one or more antimicrobials including, but not limited to, gentamicin, ampicillin, tetracycline, and erythromycin; and the microbe include one or more strains of bacteria.

In certain embodiments, the antimicrobial resistance is determined by changes in lengths of the visible bars corresponding to different concentrations of the compound against the growth of the microbe.

In certain embodiments, if a significant change in lengths between the visible bars is observed, the corresponding concentration of the compound leading to the significant change will be regarded as minimum inhibitory concentration against the microbe comparable to standard minimum inhibitory concentration (MIC) values obtained using conventional methods based on the recommendations by Clinical & Laboratory Standards Institute (CLSI) for the same compound; otherwise, it is preferably to repeat the steps described hereinabove with the same concentrations or even higher concentrations of the same compound to verify the antimicrobial resistance of the microbe.

In certain embodiments, the significant change in lengths between certain visible bars is observed from a slope of a plot of the image data for various lengths of the visible bars against different concentrations of the compound.

In yet another aspect of the present invention, there is provided a method for evaluating antimicrobial resistant microbes in a fluid sample, which includes:

loading one or more known antimicrobials into the reagent compartment of the system described herein;

using a corresponding software program installed in a paired workstation or portable device to control power on/off of and flowrate of fluid generated by the automated fluid pump of the system;

loading the fluid sample to the corresponding fluid inlet of the microfluidic-based platform;

treating the fluid sample with the one or more known compounds at different concentrations in the cell culture zone of the microfluidic-based platform under a constant temperature controlled by the thermostat for a sufficient time duration in the drug-cell incubation compartment of the system;

flushing the cell culture of microbes in the fluid sample after treating with different concentrations of the one or more known compounds from the cell culture zone via the one-piece connector to the cell sensing zone of the microfluidic-based platform by activating an antimicrobial susceptibility test function of the system through the software program of the paired workstation or portable device;

capturing images of visible bars derived from the accumulated cultivated cells of the microbes at the adaptive linear filter array of the cell sensing zone by an imaging module paired with the workstation or the portable device under an illumination by the shadowless light panel of the system;

converting the captured images into image data for various lengths of the visible bars;

comparing the image data for various lengths of the visible bars with a referenced image data to determine identity and antimicrobial resistance of the microbes in the fluid sample against the known compounds.

In certain embodiments, the one or more known antimicrobials include gentamicin, ampicillin, tetracycline, and erythromycin; and the microbes in the fluid sample include antimicrobial resistant microbes.

In certain embodiments, the treating of the fluid sample with the one or more known compounds at different concentrations in the cell culture zone of the microfluidic-based platform under a constant temperature controlled by the thermostat is for about 1 to 3 hours.

In certain embodiments of the present platform or system, a novel barcode-like cell sensor is provided in the cell sensing zone, which can be coupled with a microfluidic culture device, to generate AST results using a portable device, e.g., a cellphone (FIG. 2). This sensor utilizes an adaptive linear filter structure to concentrate suspended bacteria cells into visible miniature bars through the filtration effect of nanochannels at the channel sides. The length of each bar is proportional to the number of bacteria cultured under certain antibiotic concentrations upstream. A number of these bars corresponding to different antibiotic concentrations forms a visible barcode that can be imaged and analyzed using a portable device, e.g., cellphone, equipped with an imaging function.

The present invention also provides a fully automatic and microscope-free method for counting cells, realizing a practical, portable, low-cost, high-throughput platform for microbial assay (FIG. 30, Table 1). The AST results procured from the barcode-like cell sensor of the present invention are compared with the results obtained by the previously disclosed PP-based AST microfluidic device (microscope-based), as well as the standard minimum inhibitory concentration (MIC) values obtained using conventional methods based on the recommendations by Clinical & Laboratory Standards Institute (CLSI).

The present barcode-like cell sensor is created to meet the demand for portable AST and reduce the resources and personnel training required for operation. It makes use of an array of adaptive linear filters (or a plurality of adaptive linear filter channels) to realize a fully automatic and microscope-free method for counting a small amount of cells, which otherwise could not be seen without a microscope.

In various embodiments, after the culture period in the drug-admitted cell culture zone, bacteria are pushed to the downstream sensor zone. The suspended cells are thereby concentrated in the adaptive linear filters to form visible miniature bars, of which the length is generally proportional to the number of cells. The principle of concentration-effect by the adaptive linear filters and the fabrication details of the corresponding device or system to implement the present method will be provided hereinafter.

In certain embodiments, staining reagents can be used to make the bars clearer to see.

In other embodiments, these bars can be photographed using a portable device or device incorporated with an imaging module, e.g., a cell phone with an imaging function, and the image data obtained thereby can be used to determine MIC of a drug candidate by comparing different cell numbers after exposure to different drug concentrations. This provides a rapid phenotypic AST result without the use of sophisticated instrumentation and largely before any changes can be observed at the macroscopic scale.

In the context of environmental-related AMR testing, since a massive number of samples needs to be routinely monitored, which is very expensive and labor intensive, the present system is particularly useful. Thus, in certain embodiments, the present barcode-like cell sensor is coupled with a microfluidic culture device and through a portable device such as a cellphone to generate AST results in order to realize a practical, portable, low-cost, high-throughput platform for use in remote and resource-limited areas.

In certain embodiments, the present barcode-like cell sensor supports high-throughput operation without a massive manipulation, e.g., pipetting during broth microdilution used in conventional ASTs, which is advantageous especially when an enormous number and size of suspected samples is considered under a resource-limited condition.

In addition, detection of the unique “barcode” according to certain embodiments of the present invention realizes fully automated data-acquisition, without the need for sophisticated instruments or technician labor. By simply scanning the “barcode”, a rapid and reliable AST result can be determined through a software program such as a cell phone application.

In other words, the present device, platform and system can provide rapid screening for AMR bacteria in samples under a resource-limited condition, as well as giving a preliminary MIC value substantially in an instantaneous manner, leading to a quick determination of samples, if any, that need to be sent for advanced testing.

Other aspects of the present invention include methods of using the present platform or system in antibiotic efficacy testing for quality assurance process and drug discovery process.

Those skilled in the art will appreciate that the invention described herein is susceptible to variations and modifications other than those specifically described.

The invention includes all such variation and modifications. The invention also includes all the steps and features referred to or indicated in the specification, individually or collectively, and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF THE DRAWINGS

The above and other objects and features of the present invention will become apparent from the following description of the invention, when taken in conjunction with the accompanying drawings, in which:

FIG. 1 shows an illustration figure for the timeline of the phenotypic-based ASTs, including the conventional ASTs, automated system-based ASTs, microfluidic-based ASTs and the AST using barcode cell sensor.

FIG. 2 shows schematically and perspective image view of the present AST system according to certain embodiments of the present invention: FIG. 2A, FIG. 2A1 and FIG. 2A2 show the design of the portable AST system; FIG. 2D shows the microfluidic device which contains two parts: a cell culture zone, a barcode-like cell sensor, and a one-piece connector connecting the cell culture zone and barcode-like cell sensor, where the zoom-in schematic of which is shown in FIG. 2A2; FIGS. 2B and 2C show using a portable device, e.g., a cell phone, equipped with an imaging device, e.g., camera, adopted with a macro-lens adapter for acquiring image data of gram-stained bacteria accumulated in an adaptive linear filter array with various lengths of microbars and analyze the image data derived therefrom to determine MIC value of an antibiotic against a type of microbes according to certain embodiments of the present invention.

FIG. 3 shows the schematic illustration, simulation, and testing results of the barcode-like cell sensor: FIG. 3A schematically depicts a conventional square wave filter; FIG. 3B schematically depicts an adaptive linear filter for cell sensing and array of the adaptive linear filters forming a barcode-like cell sensor; FIG. 3B shows nanochannels among different adaptive linear filters formed through roof collapse of PDMS channel structures (lower panel) and corresponding simulation results (upper panel);

FIG. 3C shows schematic diagram of the main channel and side channels of the barcode-like cell sensor (left panel), the dimension of the main channel (inlet on the left hand side), and the simulation results of the flow velocity inside the main channel of the barcode-like cell sensor (top right and bottom right panels); FIG. 3D shows 3-D structure of an adaptive linear filter under a fluorescence confocal microscope, where the scale bar represents 10 μm; FIG. 3E shows cell accumulation inside the barcode-like cell sensor using GFP-labeled E. coli suspension; the scale bar represents 150 μm.

FIG. 4A shows the AST results of E. coli under Ampicillin after gram staining.

FIG. 4B shows the AST results of S. aureus under Gentamicin after gram staining.

FIG. 5 shows that microbes were growing on the LB agar plate by using different methods: LB agar plate for the control sample (FIG. 5A); drinking water sample by using the spread plate method (FIG. 5B); and streak plate method (FIG. 5C).

FIG. 6A shows MALDI-TOF mass spectra for the bacterial isolates isolated from the drinking water samples, where the probable species identified from the drinking water samples were Staphylococcus epidermidis.

FIG. 6B shows MALDI-TOF mass spectra for the bacterial isolates isolated from the drinking water samples, where the probable species identified from the drinking water samples were Citrobacter youngae.

FIG. 6C shows MALDI-TOF mass spectra for the bacterial isolates isolated from the drinking water samples, where the probable species identified from the drinking water samples were Staphylococcus capitis.

FIG. 7 shows the numerical simulation for the velocity distribution inside the cell sensor under different particle packing conditions (PP1 and PP2). The two groups of zoom-in panels display the orthogonal projections for the first side-channel and the side-channel at the end of the microchannel, respectively.

FIG. 8 shows the enlarged orthogonal projection for the first side-channel (1st row) and the side-channel at the end (2nd row) of the cell sensor under different particle packing conditions (PP1 and PP2) in order to show the simulation results for the velocity distribution inside the cell sensor.

FIG. 9 shows the enlarged XY-plane projection for the first side-channel (1st row) and the side-channel at the end (2nd row) of the cell sensor under different particle packing conditions in order to show the simulation results for the velocity distribution inside the cell sensor.

FIG. 10 shows the numerical simulation for the velocity magnitude of the fluid flow inside the cell sensor under different numbers of side-channels (4, 8, 16, 32). The two zoom-in panels in each channel layout display the orthogonal projection for the first side-channel and the side-channel at the end of the microchannel, respectively.

FIG. 11 shows the enlarged orthogonal projection for the side-channel at the end of the microchannel under different numbers of side-channels (4, 8, 16, 32) in order to show the simulation results for the velocity distribution inside the cell sensor.

FIG. 12 shows the enlarged orthogonal projection for the first side-channel of the cell sensor under different numbers of side-channels (4, 8, 16, 32) in order to show the simulation results for the velocity distribution inside the cell sensor.

FIG. 13 shows the enlarged XY-plane projection for the first side-channel of the cell sensor under different numbers of side-channels (4, 8, 16, 32) in order to show the simulation results for the velocity distribution inside the cell sensor.

FIG. 14 shows the enlarged XY-plane projection for the side-channel at the end of the microchannel under different numbers of side-channels (4, 8, 16, 32) in order to show the simulation results for the velocity distribution inside the cell sensor.

FIG. 15 shows the numerical simulation for the velocity magnitude of the fluid flow inside the cell sensor under different heights of the main channel (4 μm, 8 μm, 16 μm). The two zoom-in panels in each channel layout display the orthogonal projection for the first side-channel and the side-channel at the end of the microchannel, respectively.

FIG. 16 shows the enlarged orthogonal projection for the first side-channel (1st row) and the second side-channel at the end (2nd row) of the cell sensor under different heights of the main channel (4 μm, 8 μm, 16 μm) to show the simulation results of the velocity distribution inside the cell sensor.

FIG. 17 shows the enlarged XY-plane projection for the first side-channel (1st row) and the second side-channel at the end (2nd row) of the cell sensor under different heights of the main channel (4 μm, 8 μm, 16 μm) to show the simulation results of the velocity distribution inside the cell sensor.

FIG. 18 shows the numerical simulation for the velocity magnitude of the fluid flow inside the cell sensor under different widths of the main channel (8 μm, 16 μm, 32 μm). The two zoom-in panels in each channel layout display the orthogonal projection for the first side-channel and the side-channel at the end of the microchannel, respectively.

FIG. 19 shows the enlarged orthogonal projection for the first side-channel (1st row) and the side-channel at the end (2nd row) of the cell sensor under different widths of the main channel (8 μm, 16 μm, 32 μm) to show the simulation results of the velocity distribution inside the cell sensor.

FIG. 20 shows the enlarged XY-plane projection for the first side-channel (1st row) and the second side-channel at the end (2nd row) of the cell sensor under different widths of the main channel (8 μm, 16 μm, 32 μm) to show the simulation results of the velocity distribution inside the cell sensor.

FIG. 21 shows how MIC value of an antibiotic with respect to bacterial samples is determined by the slope of the accumulated bacteria obtained from AST result by the present system according to certain embodiments of the present invention.

FIG. 22 shows an example of the present barcode-like cell sensor capturing device according to certain embodiments of the present invention, and the MIC result that directly generated by using a mobile application developed in the present invention: the barcode cell sensor capturing device (FIG. 22A) and the analysis process for the accumulated bacteria inside the cell sensor (FIG. 22B); the MIC value and AST result which were analyzed by the developed mobile application (FIG. 22C1) and the details of the cell sensor's result processed by the mobile application (FIG. 22C2) and by computer assist (FIG. 22C3).

FIG. 23 shows the barcode-like cell sensor which is a downstream device for the microfluidic PP chip in the portable antimicrobial susceptibility testing (AST) system according to certain embodiments of the present invention: perspective image view (FIG. 23A) and microscopic image view (FIG. 23B) of the barcode-like cell sensor; scale bars are 5 mm and 100 μm, respectively; an enlarged view of an adaptive linear filter of the barcode-like cell sensor when in use which is accumulated with bacteria in the main channel (FIG. 23C).

FIG. 24 shows schematically the operation procedure of the barcode cell sensor of the present portable antimicrobial susceptibility testing (AST) system according to certain embodiments of the present invention: FIG. 24A shows sample loading step (step 1); FIG. 24B shows antibiotic loading stem (step 2); FIG. 24C shows bacterial loading step (step 3).

FIG. 25 shows the comparison of the performance for the bacteria accumulation under different concentrations of the bacteria [GFP-expressing E. coli] within an equal period under the flowrate at 1 μL/min. The respective optical density of the bacteria was OD0.05 (FIG. 25A), OD 0.25 (FIG. 25B), and OD0.50 (FIG. 25C); the scale bar represents 100 μm.

FIG. 26 shows the performance of the bacterial accumulation under the flowrate at 2 μL/min; the scale bar represents 100 μm.

FIG. 27 shows the AST results for bacterial isolates by using the broth microdilution method under different concentrations of antibiotics: Gentamicin (FIG. 27A) and Ampicillin (FIG. 27B).

FIG. 28 shows the general guideline for the assessment of the AST results under different situations.

FIG. 29 shows gram staining results for different types of accumulated bacteria inside the adaptive linear filter: FIG. 29A shows accumulated bacteria inside the channel of an adaptive linear filter; FIG. 29B shows gram stained result for gram-positive bacteria [S. aureus]; FIG. 29C shows gram-negative bacteria [E. coli].

FIG. 30 shows schematic illustration from a front view (FIG. 30A) and a perspective view (FIG. 30B) of the overall design of the present portable AST instrument. The instrument is composited of six different compartments include (1) a compartment for cell incubation, (2) a compartment for reagent storage, (3) a thermostat for the cell culture zone, (4) a shadowless light panel for photo capture; and the compartments for (5) peristaltic pumps and (6) main circuit board.

FIG. 31 shows the user interface for the developed mobile application according to certain embodiments of the present invention, which is applicable to obtain the MIC value and AST results by analyzing accumulated bacteria inside the barcode-like cell sensor.

FIG. 32 schematically depicts the present AST system incorporating a PP-based microfluidic AST chip and a barcode-like cell sensor, and how the system delivers an AST result as a “barcode” to be captured and analyzed by a mobile application of a portable device according to certain embodiments of the present invention.

FIG. 33 shows an actual picture of the “barcode” cell sensor with a barcode-like microchannel which can be used in conjunction with a particular mobile application to detect antibiotic-resistant bacteria.

FIG. 34 schematically depicts the barcode-like cell sensor device with different designs which are suitable for different purposes.

FIG. 35 shows an illustration figure for the integrated centrifugal microfluidic platform for the AST and “barcode” cell sensor device in circular-/disc-shaped and in glass slide format.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The present disclosure provides the following detail to assist the understanding and enabling of the present invention, and it should not be considered to limit the scope of protection to those specifics described hereinafter.

### Materials

SU-8 2050 permanent epoxy negative photoresist was purchased from Microchem Corporation, USA. Polydimethylsiloxane (PDMS, RTV615) and its curing agent was purchased from Momentive Performance Materials (Waterford, NY). Polypropylene (PP) was purchased from Orient Hongye Chemical Co., Ltd. (Shandong, China). Escherichia coli (ATCC 25922) and Staphylococcus aureus (ATCC 29213) were purchased from Shanghai Fuxiang Biotechnology Co., Ltd. (Shanghai, China). Green fluorescence protein (GFP)-expressing E. coli was obtained from Hongkai Wu from Hong Kong University of Science and Technology. Bacteria gram staining kit was purchased from BaSO Biotechnology Co., Ltd. (NTC, Taiwan). Luria Bertani (LB) broth powder, LB broth with agar (Lennox), antibiotics, including Ampicillin (AMP), Gentamicin (GEN), tetracycline (TET), erythromycin (ERY), and all other chemicals were purchased from Sigma-Aldrich. Thermal stage and syringe infusion pump used for the experiment were purchased from Xinruiqi Electronic, Inc. (LKTC-B1-T, China) and Cole-Parmer Instrument Company, LLC. (EW-74900-05, US) respectively.

TABLE 1
Material cost estimate of the present AST instrument.

| | | Present AST instrument | |
|---|---|---|---|
| | | | Material |
| Description | Supplier | Quantity | Cost (USD) |
| Instrument case | Xinyashun Co., Ltd, China | 1 ea. | $5.5 |
| Peristatic pump | Baoding Shenchen | 3 ea. | $73.5 |
| | Precision Pump Co., Ltd, | | |
| | China | | |
| Teflon tube | Shanghai Yu Fan Xiang Su, | 1 m × 1 ea. | $0.1 |
| | China | | |
| Shadowless light panel | Zfone, China | 1 ea. | $6 |
| Metal holder for | Chuang Sheng Da | 5 ea. | $15.5 |
| centrifuge tubes | Processing, China | | |
| Arduino board | Arduino, USA | 1 ea. | $23 |
| 21X Marco lens | RZX, idry, China | 1 ea. | $13 |
| DRV8825 Stepper motor | Zave, China | 3 ea. | $3.25 |
| controller | | | |
| Ceramic cartridge heater | Heng Xing Dian Re, China | 5 ea. | $3.85 |

| | |
|---|---|
| Estimated total material cost | $143.7 |
| of the present AST instrument | |

### Device Fabrication

The structure masks of the microstructures for the PP-based AST device are designed in AutoCAD 2018 and made on a laser printer (Kernel Electronics (Shen Zhen) CO., LTD., China). In certain embodiments, all the photoresist microstructures are fabricated using multilayer soft lithography. To fabricate the thermomoulding master for PP-based microfluidic AST chips, a mixture of PDMS prepolymer and curing agent (10:1 ratio) is poured onto the template and cured at 75° C. for 30 minutes. Next, the PDMS layer is carefully peeled off from the template and then attached to the glass substrate as the master to mold PP substrate. PP substrate is put on the PDMS master and sandwiched with another flat glass slide on it. Two spacers are used to control the thickness of the PP substrate. Then, this assembly is put on hot compressor and embossed at 175° C. for 1 min under 0.24 MPa. After embossing, the micro-patterned PP substrate is removed from the heat embosser and gently cooled to room temperature for a further thermo-bonding process with another PP substrate in order to assemble the PP-based AST chip. Before bonding, holes can be drilled through a flat PP plate which is for tubing connections. The PP substrate is assembled with this drilled flat PP plate in between two 5-mm thick steel plates. Two spacers equal to the thickness of the chip were used during the bonding process. Then, this assembly is put on a hot compressor and embossed at 145° C. for 2 h. After cooling down to room temperature, the whole PP-based microfluidic AST chip is obtained.

For the design of the adaptive linear filter, it is conducted by AutoCAD. In certain embodiments. the main structure of the device includes a micro-scale main channel and two side channels for filtration use. Meanwhile, dozens of nano-scale channelization channels are designed for the channelization of the liquid media. The widths of the main channel, two filtration channels and the channelization channels of the filter are 16 μm where the heights of the micro-scale channels (main channel and two side channels) are 8 μm and the nano-scale channels (channelization channels) are 0.8 μm. The so-called “barcode” cell sensor is a downstream device for the microfluidic polypropylene (PP) AST chip, where eight different adaptive linear filters corresponding to the eight different outlets of the PP MF AST chip are combined in order to form a barcode-like cell sensor.

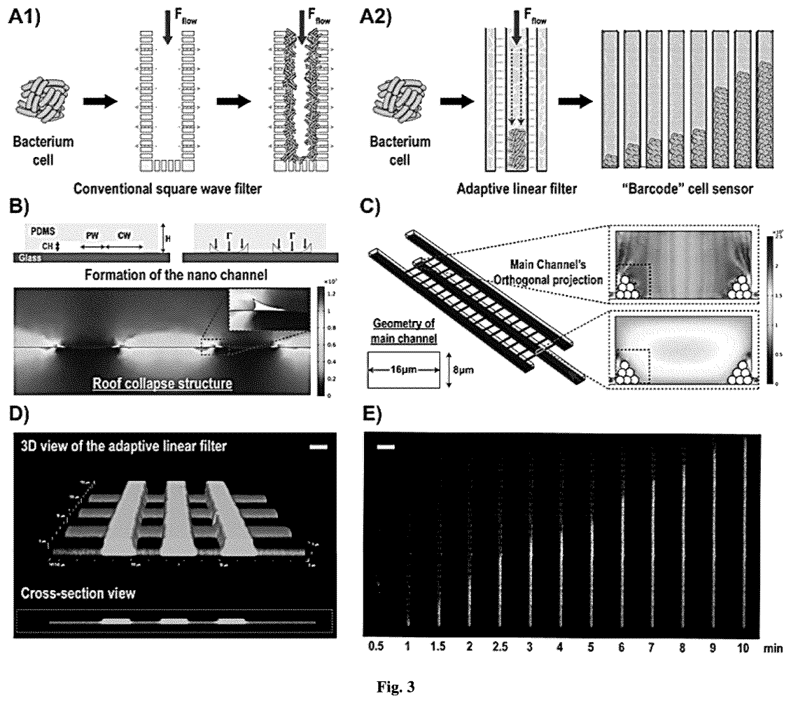

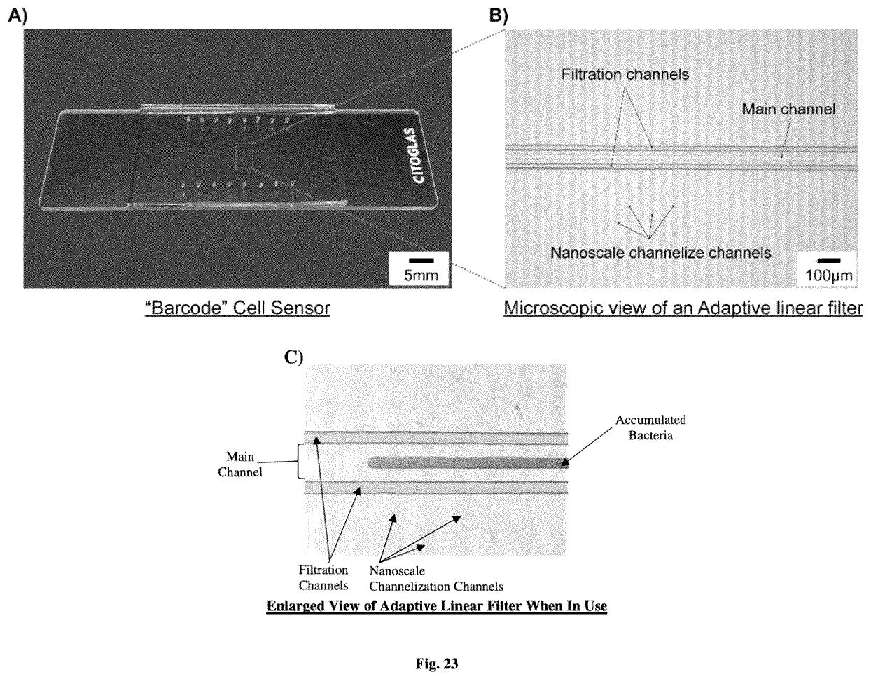

In certain embodiments, to fabricate the barcode cell sensor, a mixture of PDMS prepolymer and curing agent (10:1 ratio) is poured onto the templates and cured at 75° C. for 30 minutes. The patterned-PDMS master is then carefully peeled off from the templates. To bond the PDMS microdevice onto a glass slide, the bonding side of the PDMS master and the glass slide are treated with ozone for 5 minutes. After treatment, the PDMS and glass slide are promptly brought into contact to form an immediate irreversible bond (FIG. 3B), then the bonded PDMS device is heated at 120° C. for 20 minutes to promote bonding strength for further AST experiments (FIG. 23).

### Preparation of the Antimicrobials and Bacterial Solutions

Stock solutions of the antibiotics (AMP, GEN, TET, ERY) were prepared directly by dissolving the stock powder in deionized water at a concentration of 1000 μg/mL. An isolated colony of E. coli (ATCC 25922) and S. aureus (ATCC 29213) were suspended into 2 mL LB broth media through the use of an inoculation loop and incubated at 37° C. overnight to reach ˜5×10⁵ CFU/mL. An UV-Vis spectrophotometer (Cary 8454 UV-Vis Diode Array System, Agilent, US) was used to read the OD concentration of the bacterial suspensions.

### Real Sample Collection and Preparation

All tested samples were collected between June 2019 to November 2019 according to the guidelines from ISO 18593:2018 Microbiology of the food chain—Horizontal methods for surface sampling. Also, all of the samples were collected from the drinking fountains in government venues and university facilities, at ten different locations. As said, the surface of the outlet for the drinking fountains was tested by swabbing. In simple words, the surfaces of the drinking fountain's outlet were rubbed by a single use sterilize cotton swab and swished into the Lysogeny broth (LB) broth solution for 1 minute to prepare the sample solutions. Collected samples were kept at 4° C. during the transportation and enriched for 12-14 hours under 37° C. After the enrichment for bacteria, the sample suspensions were spread and streaked onto the LB agar for further bacterial isolation and AST uses. For complicated samples, simple filtration followed by a pre-incubation inside a selective growth medium was suggested. If an ID is needed, dilution plating can be used. Besides, antifungals and algal inhibitors can also be added into these samples to against and inhibit the growth of fungi, yeast, and algae.

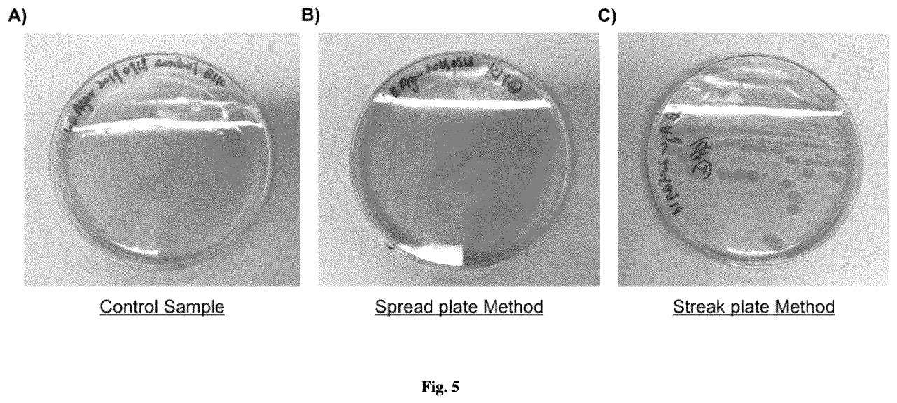

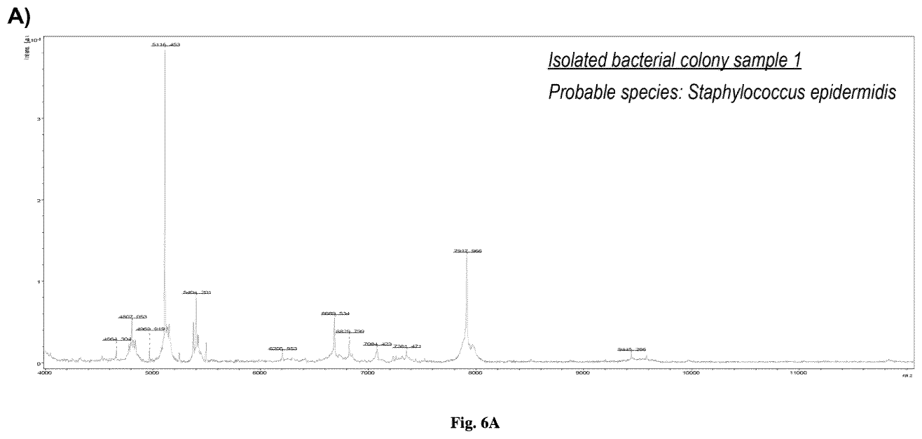

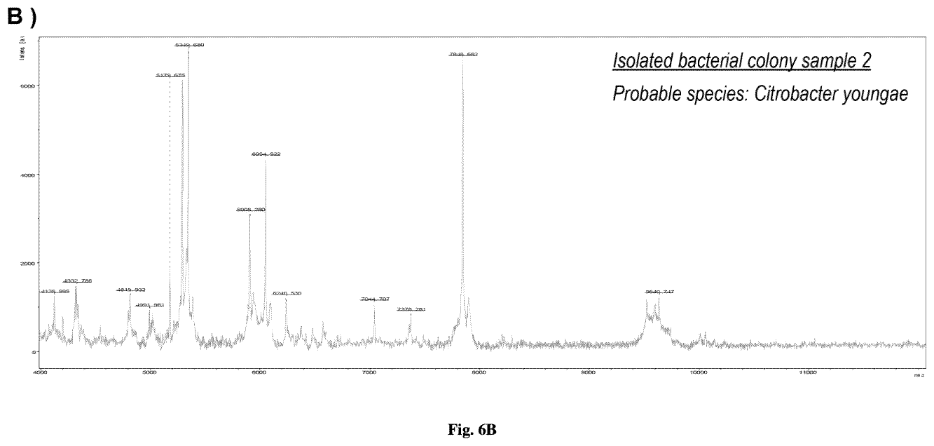

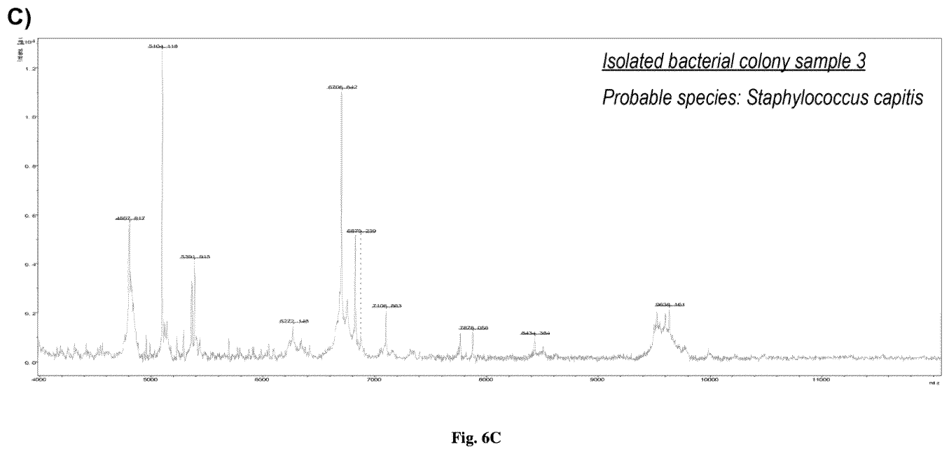

All tested samples were collected from drinking fountains at ten different locations in various government venues and university facilities. Sample containers were kept sterile before use in sample collection; the collected samples were kept at 4° C. and analyzed within 24 hours of collection. Sample collection was conducted 3 times for each sampling site and the antimicrobial susceptibility of each sample was tested 3 times. (FIG. 5) Bacterial identification for the real samples was made through the use of Matrix-Assisted Laser Desorption Ionization Time of Flight Imaging Mass Spectrometry (MALDI-TOF IMS). 1 uL of the sample was pipetted onto an Indium tin oxide (ITO) coated glass slide and allowed to dry on a hotplate; the ITO slide with bacteria was then analyzed by MALDI-TOF IMS immediately following matrix application. (FIG. 6, Table 2)

Ten different isolates have been isolated from the drinking water samples for further test of antimicrobial susceptibility, where MALDI-TOF-MS has been used for the species identification of the bacteria before the test. Eight out of ten isolates have been successfully identified by searching against the UniProt with the corresponding MS spectrum, where the source code of the UniProt is available at https://github.com/dipcarbon/BacteriaMSLF. Three possible bacterial species have been identified in those eight isolates, which are Staphylococcus epidermidis, Citrobacter youngae, and Staphylococcus capitis.

Staphylococcus epidermidis (FIG. 6A) and Staphylococcus capitis (FIG. 6C) are coagulase-negative species (CoNS) of Staphylococcal strains which usually produce a slimy biofilm/even a multi-layered biofilm that make the antibiotics become less effective. S. epidermidis and S. capitis may cause infection or even sepsis, and some of the widespread resistance stains have been reported in different reports already. On the other hand, Citrobacter youngae (FIG. 6B) is an opportunistic pathogen of humans and associated with a range of infections like urinary tract infections (UTIs), gastroenteritis, septicemia etc.

TABLE 2
Bacteria identification results of the bacterial isolates which
isolated from the drinking water samples by using MALDI-TOF-MS.

| Probable species | Isolate 1 | Isolate 2 | Isolate 3 | Isolate 4 | Isolate 5 | Isolate 6 | Isolate 7 | Isolate 8 |
|---|---|---|---|---|---|---|---|---|
| Staphylococcus | + | | | | + | | | |
| epidermidis | | | | | | | | |
| Citrobacter | | + | | | | + | + | |
| youngae | | | | | | | | |
| Staphylococcus | | | + | + | | | | + |
| capitis | | | | | | | | |

+ stands for positive.

### Antimicrobial Susceptibility Experiment

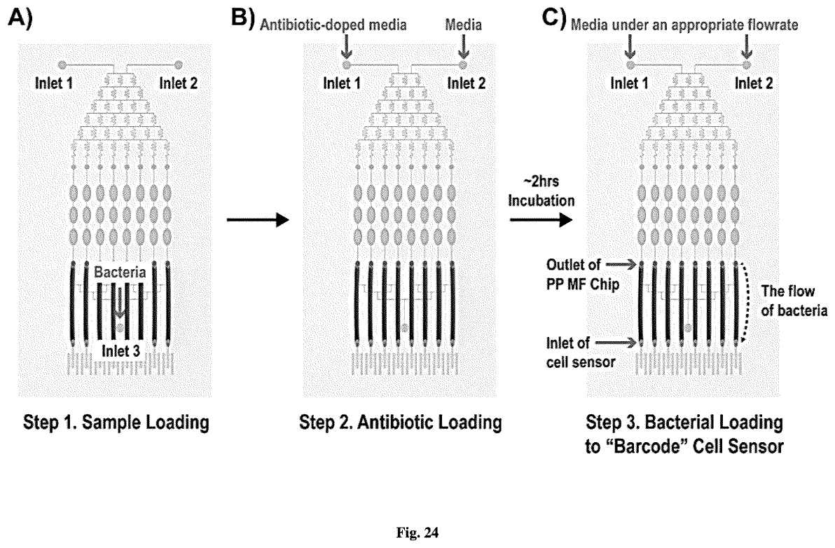

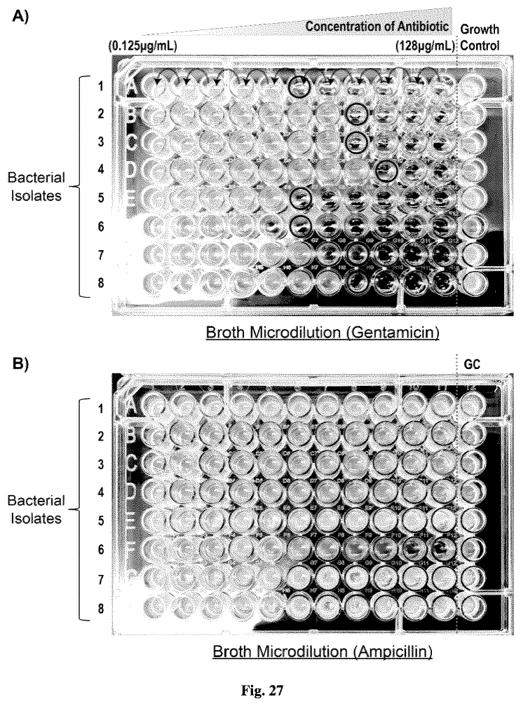

To conduct an AST on the PP-based AST chip, bacteria were pre-activated inside an incubator for 15 minutes at 37° C. before use. At the beginning of a test, the bacteria are injected through Inlet 3; the injection of the bacteria was monitored under the fluorescence microscope (FIG. 24A). After the bacteria were inoculated into the cultivation chambers, antibiotic-doped media and media alone are injected into inlet 1 and inlet 2 at a rate of 1 μL/minute, respectively (FIG. 24B). The AST chip was then incubated on a thermostat platform at 37° C. for 2 hours. To assess AMR, the bacteria inside the culture chambers of the PP AST chip were flushed into the barcode cell sensor at a rate of 1 μL/minute. Gram staining agent was used during the flushing process to make bacteria inside the barcode cell sensor visible be captured by a cell phone camera. For comparison, AST was also performed using the traditional broth microdilution method as shown in FIG. 27; the MIC values of the bacteria were assessed as the lowest concentration of antibiotics with no visible growth of bacteria.

The broth microdilution (BMD) testing of the samples was performed exactly the same according to the approved standard from CLSI and the procedures described in Nature Protocol. To perform the BMD test, both LB broth medium and the antibiotics were added into the 96-wells microplate. By using a two-fold dilution, a concentration gradient of the antibiotic is generated from 128 μg/mL to 0.125 μg/mL. The bacteria isolates identified inside the samples have been enriched under 37° C. for 8-12 hrs in order to achieve 5×10⁶ CFU/mL before the test, 10 uL of the bacterial suspension was inoculated into the broth to achieve a final concentration of bacteria in 5×10⁵ CFU/mL. The inoculated microplates were incubated at 37° C. for 16-20 hrs for further inspection. To assess the MIC values for the samples, the microplates were examined visually and the lowest concentration of the antibiotic which prevents visible growth of the bacteria was recorded as the MIC value of that sample. Meanwhile, Mueller Hinton broth was also used to perform the BMD for cross checking purpose; both of the AST results are consistence with the MIC quality control range recommended by CLSI.

According to the AST results of the present disclosure, all those identified isolates (isolates 1-8) have shown susceptibility to Gentamicin where all of them are resistant to Ampicillin at the same time. Meanwhile, the MIC values are tested with mixed bacteria samples as a reference to prove the feasibility of multi-bacterial AST screening. For the AST results of the mixed bacteria sample, the MIC value of the sample is 4-8 μg/mL in gentamicin (FIG. 27A). On the other hand, the mixed bacteria sample has shown resistance to ampicillin (FIG. 27B).

### Computational Fluid Dynamics (CFD) Simulation

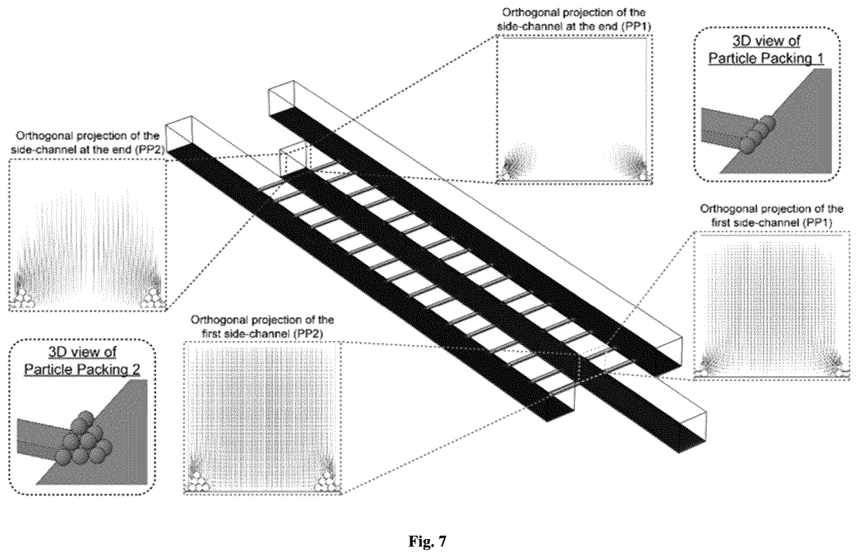

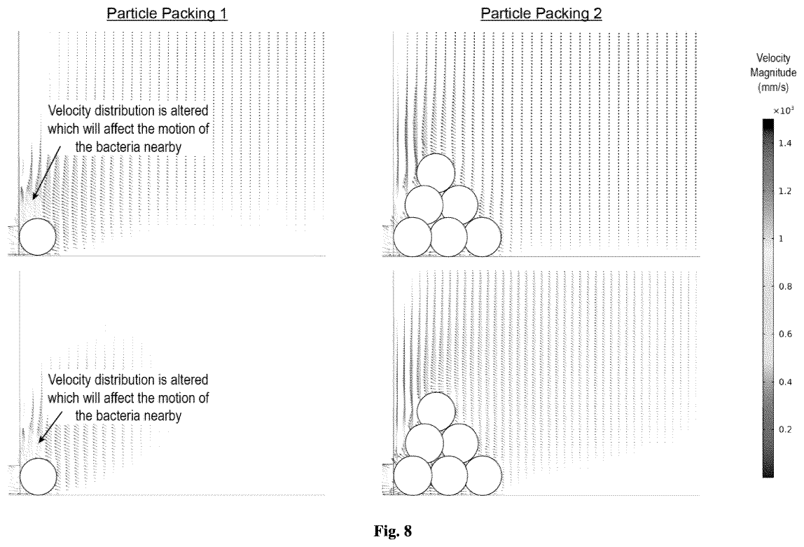

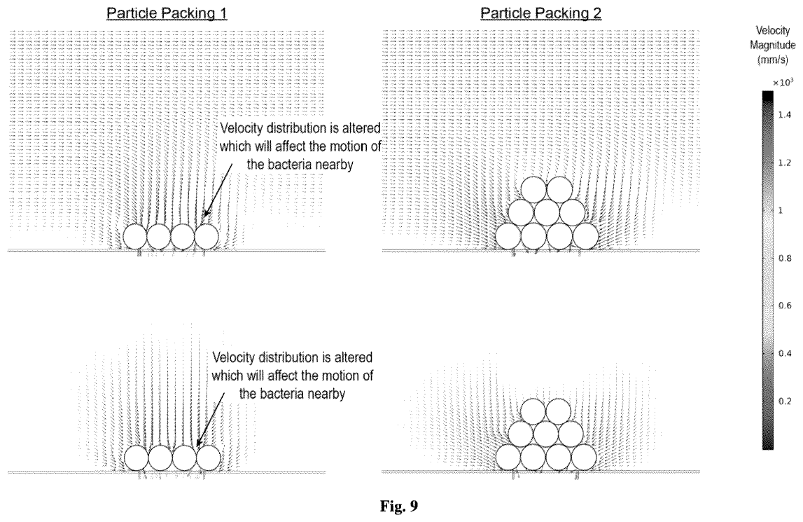

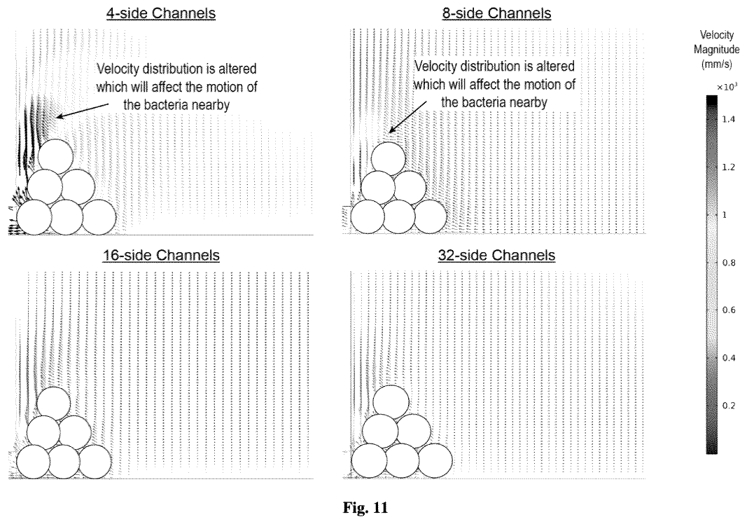

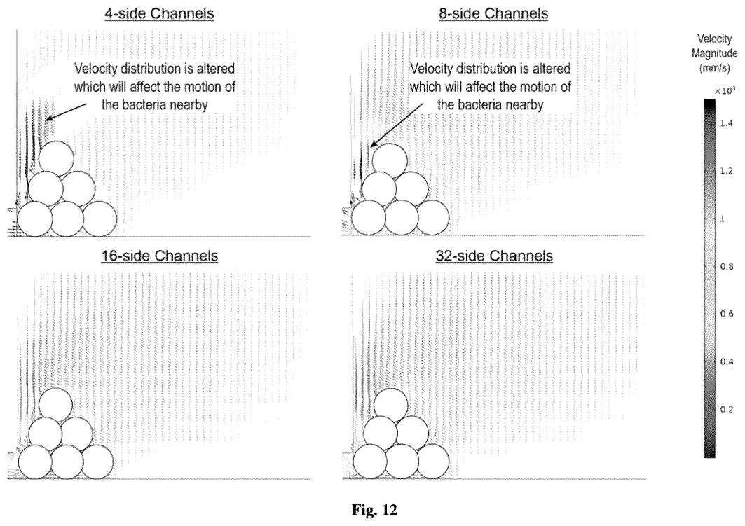

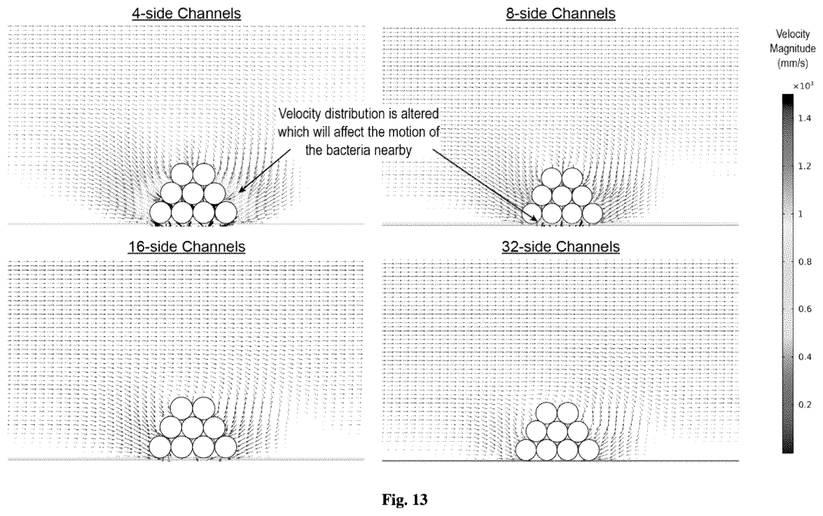

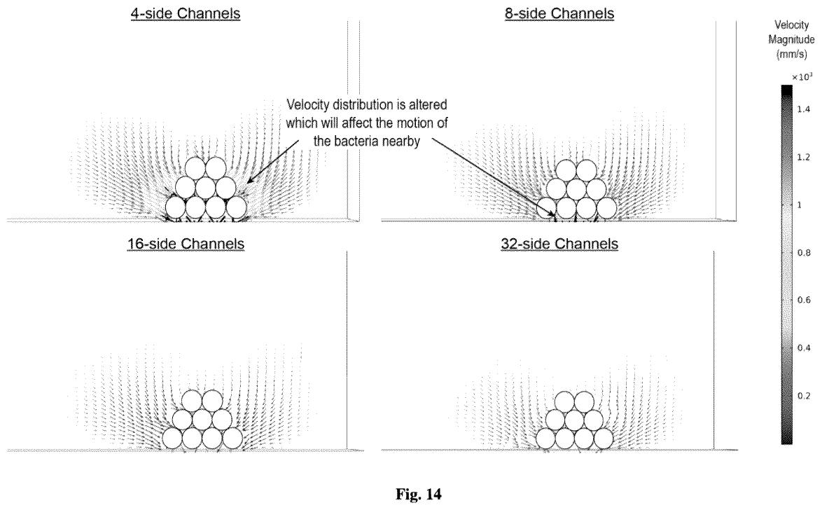

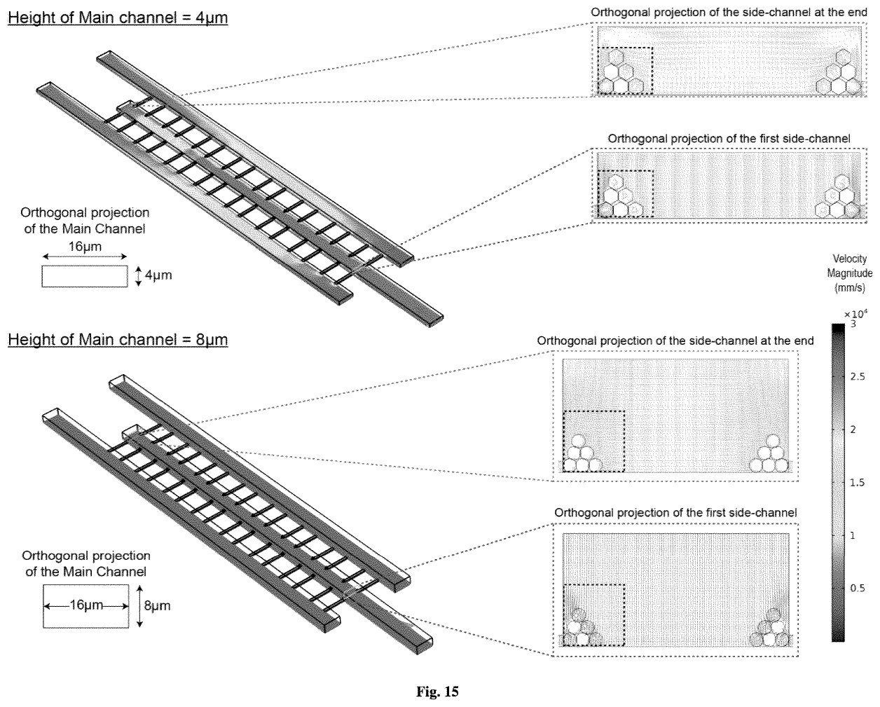

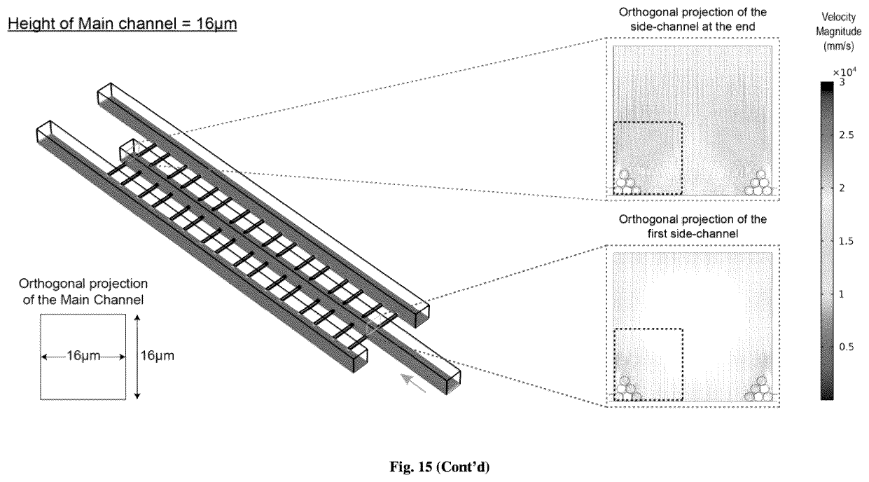

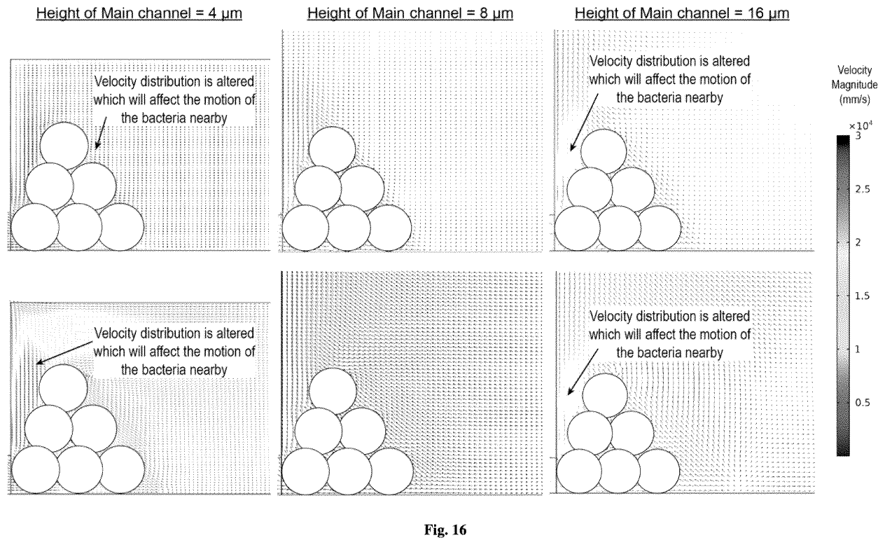

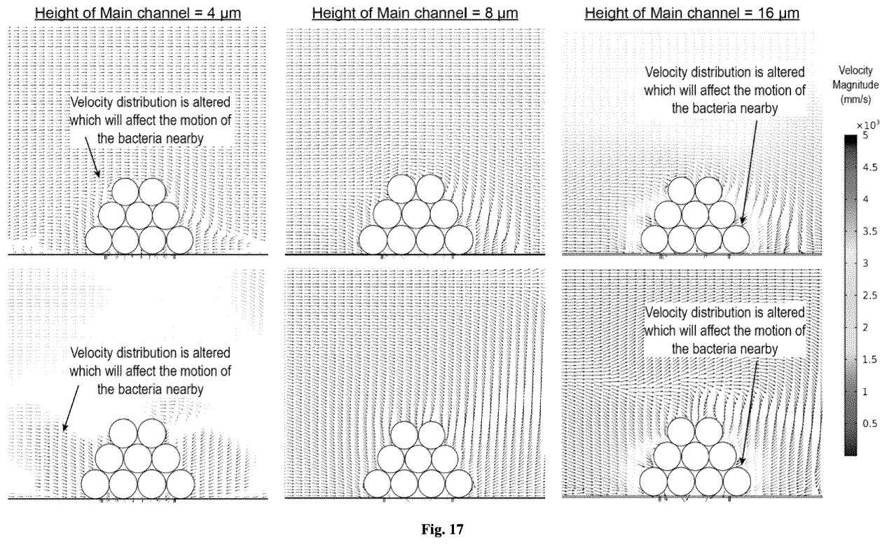

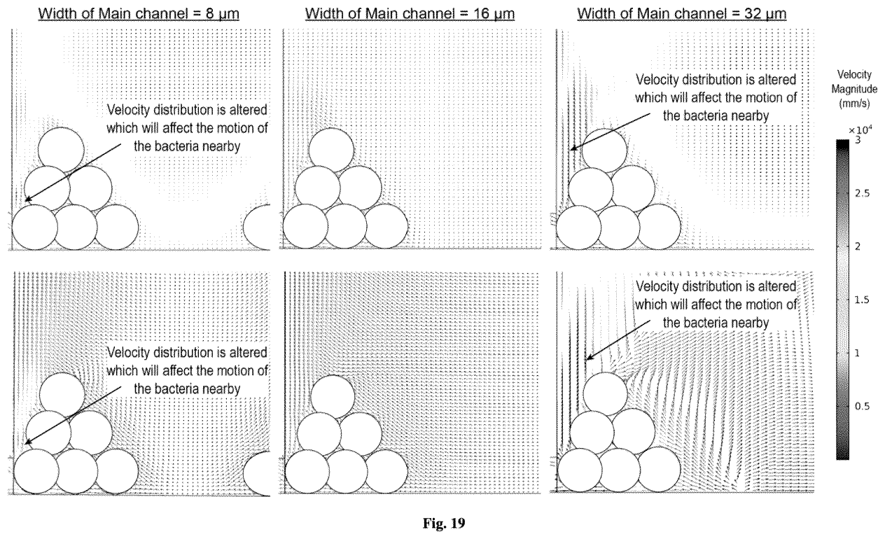

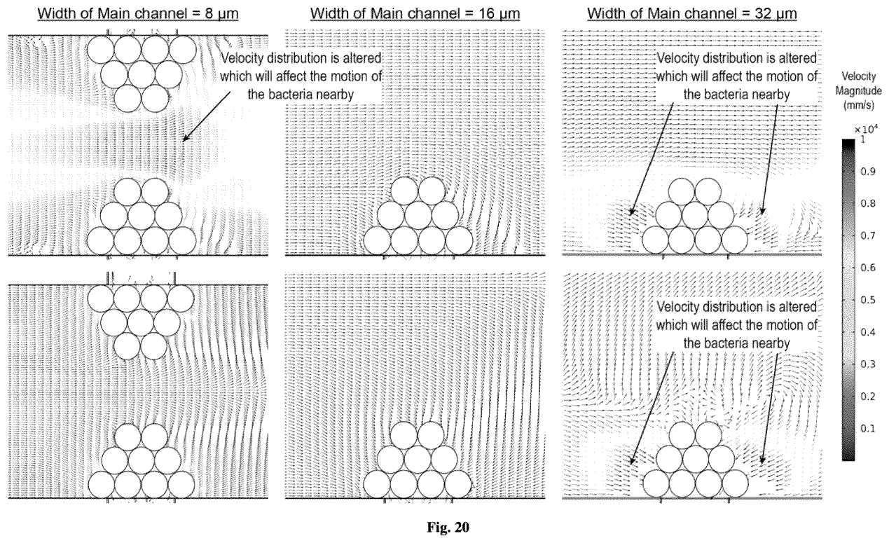

Computational fluid dynamics simulations were conducted using COMSOL Multiphysics 5.4 (COMSOL Inc., MA) to investigate the formation of the roof collapse structure that generates the nanochannel, as well as the velocity magnitude of fluidic flow inside the microfluidic channel under different types of particle packing and channel designs. (FIGS. 7-20) The 3D models of the adaptive cell sensor with nano-channelized channels were established in AutoCAD 2019 and meshed into triangular elements for COMSOL simulations. All of the simulations were based on the incompressible Naiver-Stokes equation and the inner surface of the microfluidic channel was assumed to be non-slip.

COMSOL Multiphysics 5.4 (COMSOL Inc., MA) was used to investigate the formation of roof collapse structure and also the velocity distribution inside the microfluidic cell sensor. Also, in order to simulate the fluid dynamics inside the cell sensor, the following assumptions are made: 1. the viscosity and density of the sample fluid is constant, 2. the inner boundary layer of the cell sensor is assumed to be non-slip, 3. the flow rate of the sample fluid is fully developed at the inlet of the model and obey the incompressible Naiver-Stokes equation and, 4. There have no gravitation or other volume forces that affect the sample fluid. All 3D models are established by AutoCAD 2019 and meshed into triangular elements for COMSOL simulations.

$$\\ \begin{matrix}\frac{\partial u}{\partial t} + ( u \cdot ∇ ) u = - \frac{1}{ρ} ∇ p + v {∇}^{2} u + f & ( 1 )\end{matrix}$$

$$\\ ∇ . u = 0$$

where f is an external acceleration field [m·s⁻²], p is the density [kg·m⁻³], p is the pressure field [pa], v is the kinematic viscosity [m².s⁻¹], u is the fluid velocity field [m·s⁻¹], and t is time [s].

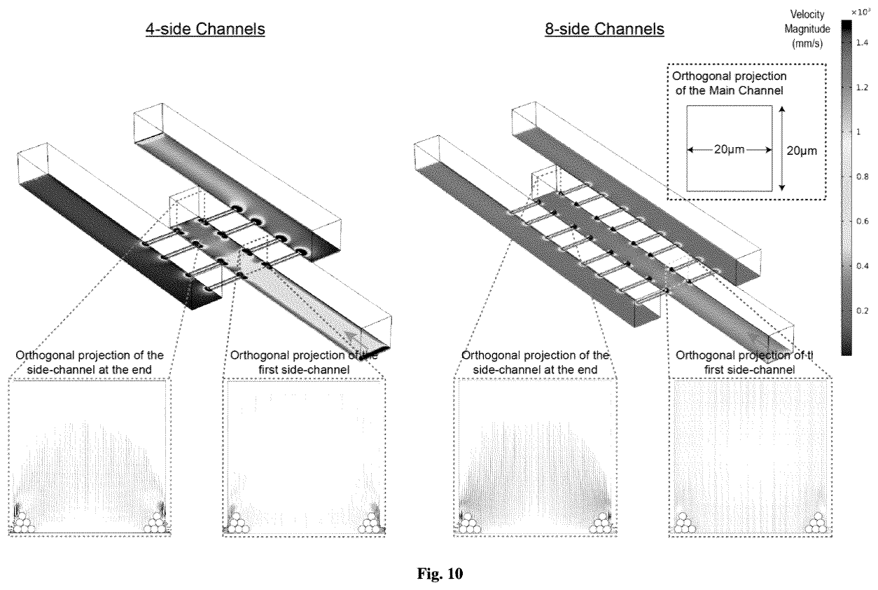

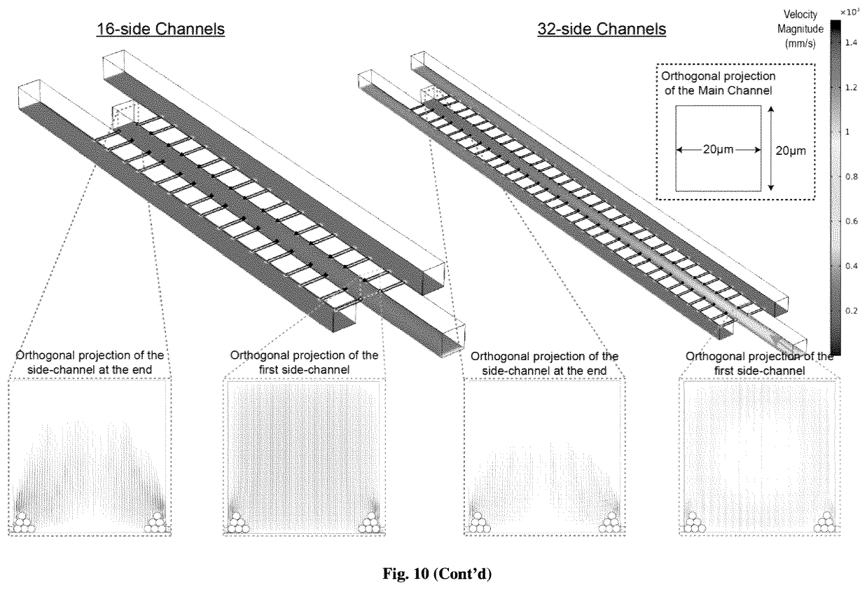

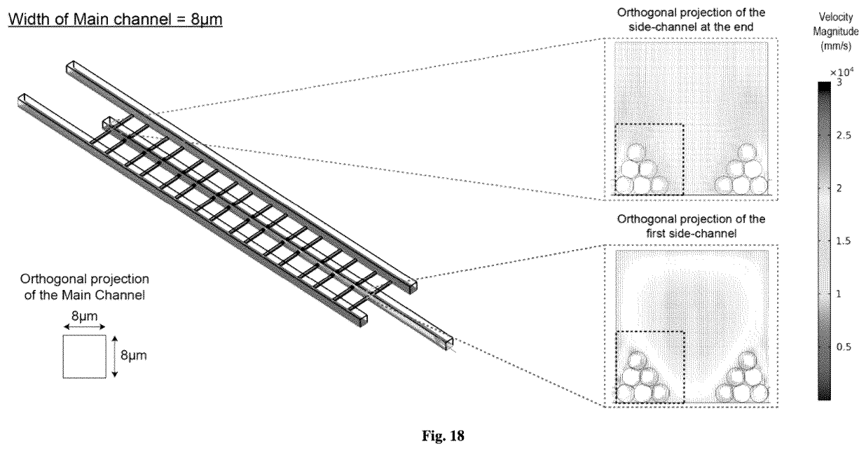

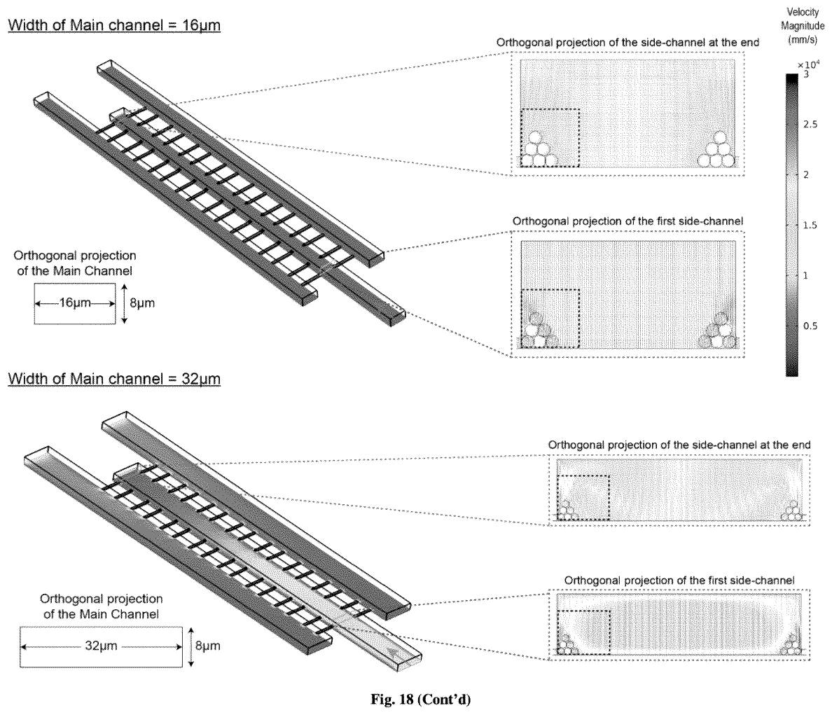

To better simulate the real situation of the velocity distribution inside the cell sensor during bacteria accumulation, different particle packing conditions have been tested before further simulation is done. In order to facilitate further simulations, all the bacteria particles were assumed to be sphere-shaped and packed with face-centered cubic packing according to the simulation results obtained and shown in FIGS. 7-9. The variables within simulations are mainly based on the differences in side-channels number (FIGS. 10-14), width (FIGS. 18-20), and also the height (FIGS. 10-17) of the main channel.

### Image Collection and Analysis

As said, the bacteria samples have been flushed into the “barcode” cell sensor after 2 hours incubation, the accumulated bacteria bars with different lengths will be formed. Also, by calculating the slope within the accumulated bacteria bars, a sharp turning point will be shown to indicate the range of the MIC values. A so-called turning point is in between the bars which have a significant difference in lengths for the accumulated bacteria, and the difference in accumulated length represents a significant change in the number of bacteria. According to the CLSI, MIC is defined as the lowest concentration of a given antimicrobial which inhibits the growth of a particular bacteria. Thus, the MIC value of the samples can be assessed by looking at the point (in a specific concentration of a given antibiotic) of the bacterial growth with significant differences using the comparison of the log [slope] values.

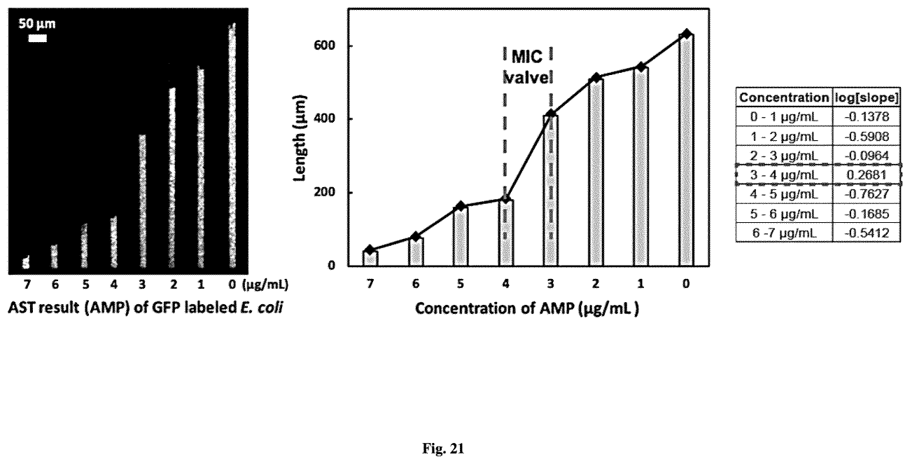

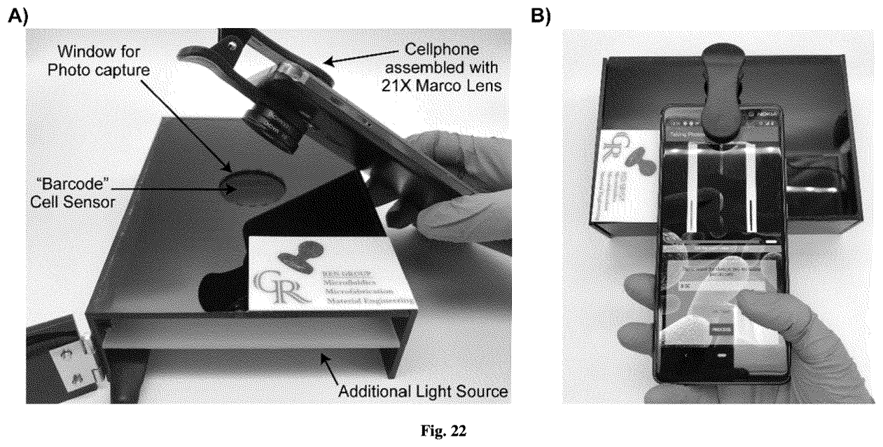

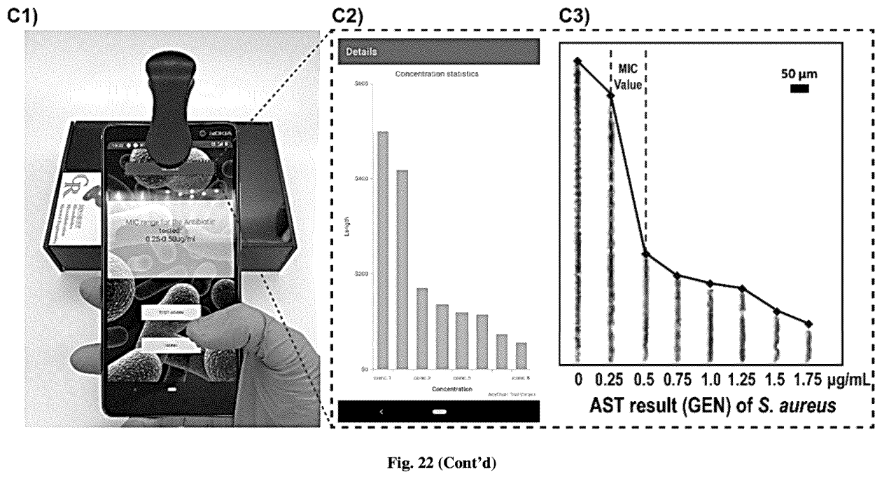

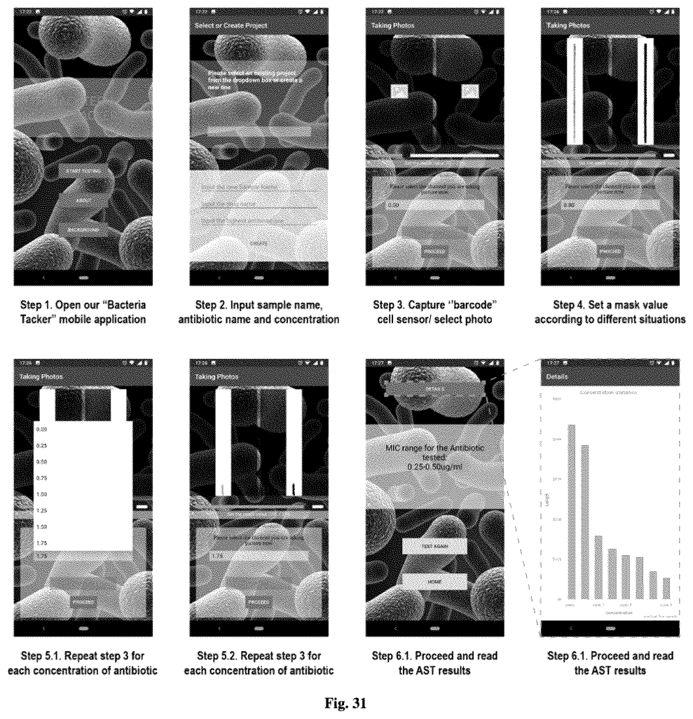

The PP-based microfluidic AST chip was placed on an inverted fluorescence microscope Eclipse Ts2R (Nikon, Japan), equipped with an Infinity 3 s digital camera (Lumenera Corporation, Canada), to capture pictures. For the “barcode” cell sensor, images were captured after gram staining through use of a cell phone equipped with a 21× macro-lens (olloclip, USA). The MIC value was assessed by both bacterial cell number and morphology change after incubation inside the PP-based AST chip, as well as by the accumulated length of bacteria inside the cell sensor. Pictures were post-processed with the ImageJ 1.52 program (FIG. 21) and also on the mobile application (FIG. 22 and FIG. 31) as provided for further analysis and statistics.

### The Overall Design of the Present AST System

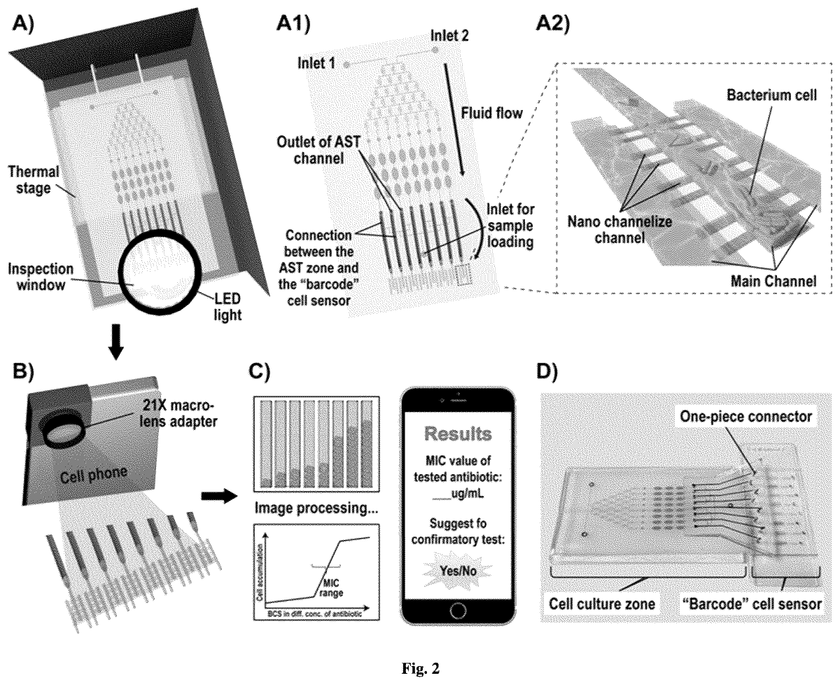

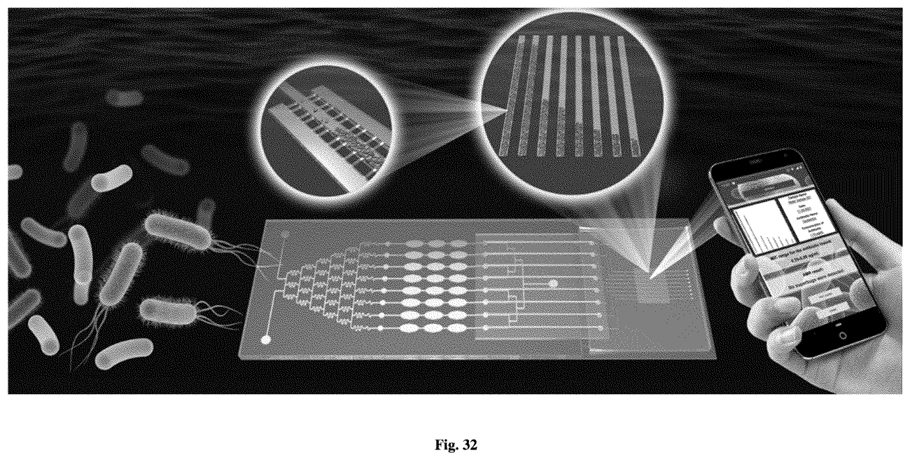

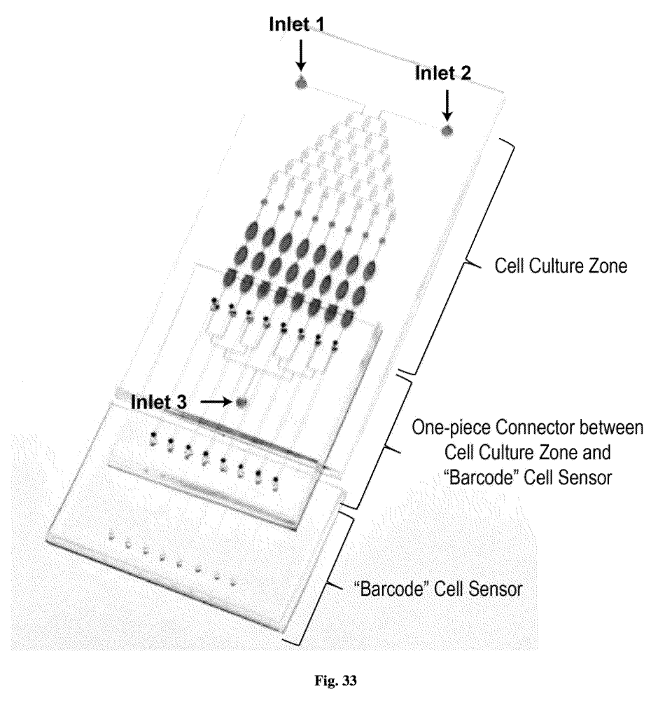

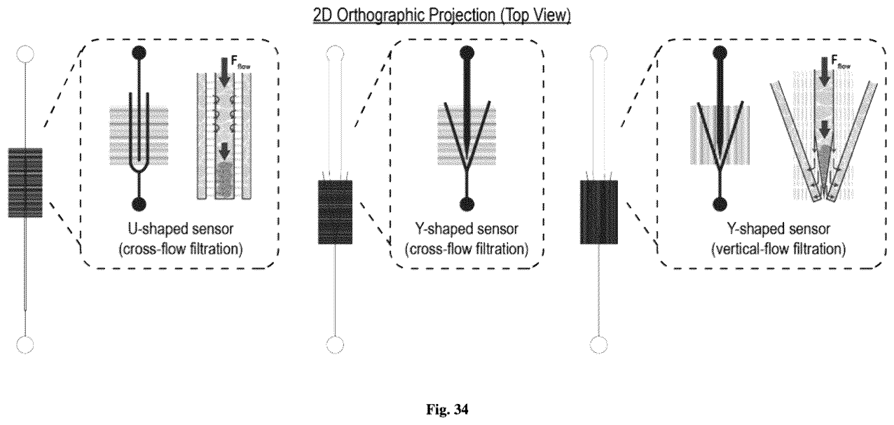

The present automatic AST system includes two main parts: a microfluidic cell culture zone for exposing bacteria to the tested drug, and a barcode-like cell sensor zone (FIGS. 2A1, 2A2 and 2D) for microscope-free cell counting. The system is a microfluidic device comprising an enclosure, a thermal stage, a light source (such as LED light), and an inspection window, where the cell culture zone includes a Christmas tree-structured drug concentration gradient generator, followed by micro-chambers with deepened microwells for bacterial culture disposed downstream to the drug concentration gradient generator. A bacterial specimen will be incubated in the drug-admitted culture zone first by loading to one of the inlet ports at the cell culture zone, i.e., Inlet 3 as shown in FIG. 24, while drug-containing cell culture media and cell culture media alone are injected from Inlet 1 and Inlet 2 as shown in FIG. 2A1, respectively, and then flushed into the cell sensor zone to complete the AST. In certain embodiments, the bacteria are cultured in the drug concentration gradient for 1-3 hours before being pushed into the cell counting zone by using an increased flow rate. Bacteria will then accumulate inside the adaptive linear filters in the barcode-like sensing zone, and after gram staining the results can be captured and analyzed by using a portable device such as a cell phone (FIG. 2C) equipped with a macro lens adapter (FIG. 2B). FIGS. 2D and 34 shows images of the actual microfluidic device fabricated according to various embodiments of the present invention, which contains a cell culture zone and a barcode cell sensor connected by a one-piece connector. A key feature of the present barcode-like cell sensor is that suspended bacteria cells flowing through the adaptive linear filter will accumulate at the downstream end of the adaptive linear filter channel; the accumulated cells will then generate a visible bar, the length of which is proportional to the number of cells in suspension, so that a microscope-free cell counting function is achieved (FIG. 3A2). Since multiple cell-culture channels are employed for AST purposes in the cell culture zone, one cell-counting filter is connected to each of the channels, and these filters are arranged in parallel to generate a barcode-like structure (FIG. 3, FIGS. 23A-23B, FIG. 32 and FIG. 33). A cell phone can be used to photograph this barcode-like structure and generate an analysis of the relative lengths of different bars, which provides information on the MIC value for the tested drug against the tested sample. This allows fast and microscope-free counting of a small amount of bacteria, thus realizing rapid, easily-accessible, and portable AST.

### The Cell Culture Zone

The microfluidic cell culture zone of the present AST system is used to perform a phenotypic AST, wherein the bacteria are exposed to various concentrations of antibiotics and show different proliferation rates. As shown in FIG. 2A, the module includes two parts: a plurality of diverging fluid channels (Christmas tree-structured) being a concentration gradient generator for feeding antibiotics and a number of deepened microwells for the bacteria culture. Fluid flow is needed to provide the cell culture with nutrient support, as well as to generate the antibiotic concentration gradient in the cell culture zone. The deepened microwell structure in the middle region were designed to achieve more suitable shear rate and thus facilitate the attachment of bacteria during the culture period. A fundamental uniqueness of this 3-D chamber design is that it has a vertically layered flowrate distribution while the conventional designs for reducing shear rate often use planar layout. The present design can utilize the sedimentation effect of bacterial cells, which spontaneously stabilize them onto the bottom surface of the deepened microwells, where the shear stress thereof is lower than that of the majority of the chamber. A benefit of such design is that a high flowrate in the main channel is allowed during the cell loading process, allowing rapid and high-throughput operation. After the bacteria have been pre-loaded into the deepened microwells, the antibiotic-doped media and media alone are injected into inlets 1 and 2, respectively (FIG. 2A1 and FIGS. 24A-24C). A linear antibiotic concentration gradient ladder will thus be generated on the device and affect cells cultured in the deepened microwells. In addition, by changing the design and the fluidic control for the part of the concentration gradient generator, a logarithmic concentration gradient can be generated instead of the linear one.

The cell culture zone is made of a thermoplastic material called polypropylene (PP). There are two reasons for selecting PP for making this module. Firstly, PP has been employed in a broad range of commercial and industrial implementations due to its solvent compatibility and anti-fouling property. Compared to PDMS, PP is cost-efficient, solvent-compatible, reusable, free from the absorption of hydrophobic drug molecules and water evaporation material, which can address the problem of drug concentration deviation in a PDMS channel, thereby providing more reliable AST results. Secondly, the design of the present system requires the cells to stay in the culture chamber during the culture period and move to the downstream sensor channel afterwards. According to some previous findings, cells cultured in a PDMS channel will attach to the bottom of the channel after the culture period and cannot be flushed downstream afterwards. In contrast, a PP channel allows temporary settling of cells and subsequent movement downstream by simply increasing the flowrate.

As discussed above, conventional methods obtain AST results by observing macroscopic changes in the sample, such as the formation of colonies or a shift in turbidity, which take more than ten hours to complete. In a previous study, it was demonstrated that by monitoring the change of individual cells, on-chip AST results could be obtained within 2-3 hours. Although this strategy can acquire AST results much faster than the conventional AST methods, it relies on the inspection of single cells with a microscope, which is resource-dependent and labor-intensive. To address this bottleneck for implementation in routine on-site screening, especially under a resource-limited condition, the barcode-like cell sensor is thereby provided in the present invention.

### Barcode-Like Cell Sensor

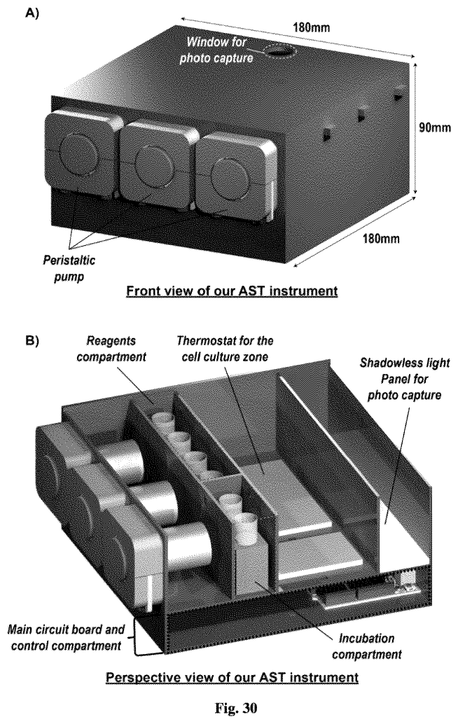

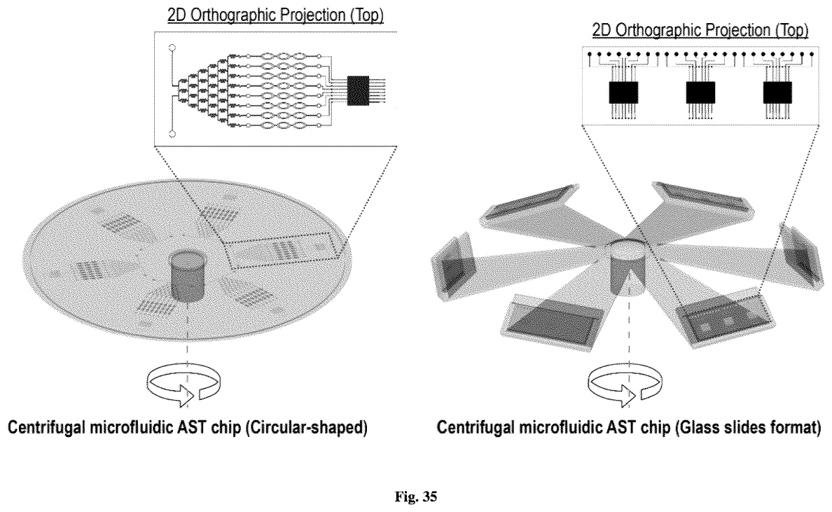

The present barcode-like cell sensor is provided to meet the demand for portable AST and reduce the resources and training required for operation. It makes use of an array of adaptive linear filters (a plurality of adaptive linear filter channels) to realize a fully automatic and microscope-free method for counting a small amount of cells, which otherwise could not be seen without a microscope. After the culture period in the drug-admitted cell culture zone, bacteria are pushed to the downstream sensor zone (FIGS. 2A1 and 2A2). The suspended cells are concentrated in the adaptive linear filters to form visible miniature bars, of which the length is generally proportional to the number of cells (FIGS. 3A2 and 3E). The principle of concentration-effect by the adaptive linear filters and the fabrication details are discussed in later sections. Staining reagents can be used to make the bars clearer to see; these bars can be photographed using a cell phone and used to compare different cell numbers after exposure to different drug concentrations (FIGS. 2B and 2C). This provides a rapid phenotypic AST result without the use of sophisticated instrumentation (FIGS. 30A and 30B, Table 1) and largely before any changes can be observed at the macroscopic scale. For example, in environmental-related AMR testing, a massive number of samples need to be routinely monitored, which is very expensive and labor intensive. In such circumstances, the present system is particularly useful. The barcode-like cell sensor can be used integrally or independently, depending on the purpose of detecting or sorting cells. FIG. 35 illustrates an example of an integrated centrifugal microfluidic platform for the AST and the barcode-like cell sensor in circular- or disc-shaped and in glass slide format. The disc-shaped AST centrifugal microfluidic platform integrates both the cell culture and cell sensing zones into the same platform, mainly for AST purpose. In contrast, the glass-slide AST centrifugal platform integrates the cell sensing zone only, such that it can be used with different drug-cell and cell-cell interaction studies including AST and cell sorting, e.g., CAR-T cell sorting. Also, by changing the dimension (e.g., width and height of the main and side channel, number and the arrangement of the channelization channels, etc.) of the design, a variety of cell types which are compatible with the present barcode-like cell sensor for either detecting or sorting purpose includes bacteria, mammalian cells, yeast, and plant cells, etc. FIG. 34 depicts three different designs of the barcode-like cell sensor according to various embodiments of the present invention, including one U-shaped sensor (cross-flow filtration), and two Y-shaped sensors (cross-flow and vertical-flow filtrations, respectively). Different sensor (filter) designs are used for different cell types, subject to fluidic characteristics inside the sensor such as shear stress and velocity of the fluid in the presence of the cells. With different designs of the sensor, the barcode-like cell sensor can be used for AST and cell analyses of a wide variety of cell types including bacteria, mammalian cells, yeast, plant cells, etc., especially in case where the cells may be influenced by the sorting process after cell sorting.

### Operation Procedure for the Barcode-Like Cell Sensor in the Present AST System

FIG. 24 illustrates the details for the operating procedure for the barcode cell sensor in the present AST system. As mentioned, the design of the present AST system contains two parts: the first part is a microfluidic (MF) PP AST chip used for bacterial culture and the second part is a barcode-like cell sensor which is used to inspect MIC value of the cultured bacteria. In the beginning, the pre-cultivated sample which contains bacteria inside was injected into the PP AST chip through the Inlet 3, and the process of injection was monitored under the inverted fluorescence microscope (FIG. 24A). After the bacteria were injected inside the cultivation chambers of the PP AST chip, the antibiotic-doped media and the media were injected into the chip through the inlet 1 and inlet 2, respectively (FIG. 24B). A concentration gradient of the antibiotic was generated due to the unique design of the PP AST chip, bacteria inside the deepened micro-chambers were further cultured at 37° C. on a thermostat platform for 1-3 hours.

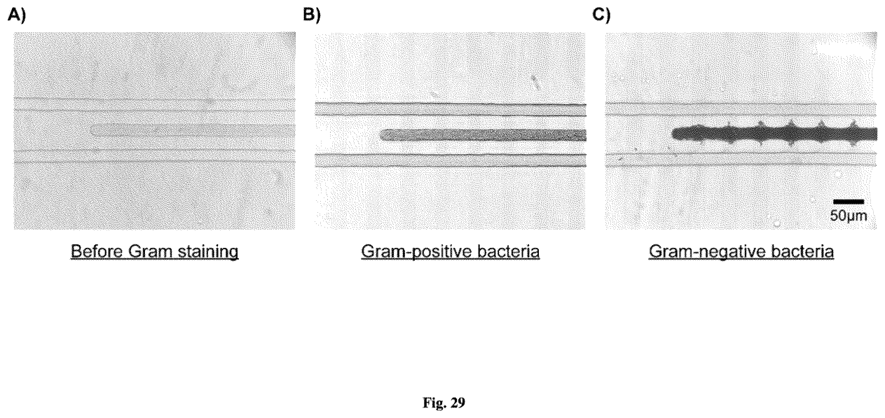

After the bacteria were cultured inside the drug concentration gradient for 1-3 hours, the bacteria inside the deepened micro-chambers were then pushed into the downstream barcode cell sensor for cell counting and MIC assessment by using an increased flow rate (FIG. 24C). Eight different outlets of the PP AST chip were correspondence to eight different inlets on the “barcode” cell sensor, and the bacteria flushed into the cell sensor will accumulate and generate a visible pattern for inspection. MIC values of the samples can be assessed by comparing the length of the accumulated bacteria inside the sensor under different concentrations of antibiotics by simply using an inverted microscope. In order to realize and improve the portability and convenience of the present AST system, the MIC values can also assess by using a mobile phone that equipped with a lens adapter after a simple gram staining process to increase the visibility of the accumulated bacteria (FIG. 29A-29C).

### Adaptive Linear Filter for Sensing Cell Number

A key requirement of the barcode cell sensor is a linear filter that can concentrate bacteria in a suspension into a densely packed bar so that the number of the cells in suspension can be generally estimated by the length of the formed bar. There are generally two hurdles to the realization of such a “cell number ruler”. The first is the small size of bacteria: as the diameter of bacterium cells is only 0.5-1 μm, the reported microfabricated filtration structures on microchips are often hard to scale down for bacteria due to technical limitations. Fabricating pre-designed nanochannels could be costly and impractical for the production of cost-efficient devices. Instead, reported on-chip bacteria filtration was often realized with nanopore membranes, which are hard to further microfabricate into special hydrodynamic designs and inappropriate for the transmittance-based imaging needs of the present barcode sensor. Therefore, a cost-efficient strategy to produce nanopore arrays on the sidewall of the channels is required.

In the present disclosure, this challenge is addressed by exploiting the spontaneous deformation and self-adherent property of an elastomer, PDMS. With low elastic modulus (G≤1 MPa), a PDMS channel with low aspect ratio is easy to collapse, and the resulting structure can be stabilized by the self-adhesion effect, and even be fixed after heating. The schematic of the roof collapse structure used in the present invention to form nanoscale channels is shown in FIGS. 3B and 3D. The so-called roof collapse structure has been observed and interpreted in several different reports. Briefly, the formation of roof collapse can be attributed to both external compressive load and the internal adhesion between the PDMS and glass substrate. The width of the punch (PW) and the height of the channel (CH) are two of the most important determinants in the formation of the roof collapse structure. In the present invention, due to the small aspect ratio of channel height to punch width (CH/PW«1), the roof of the PDMS channel will deform and contact the glass substrate due to interfacial adhesion. In this regard, the driving force of adhesion between two substrates can be described as the equation below, where y is assumed to be adhesion energy between PDMS and the glass substrate:

$$\\ \begin{matrix}γ = {γ}_{PDMS} + {γ}_{{SiO}_{2}} - {γ}_{int} & ( 2 )\end{matrix}$$

Additionally, the strength of the adhesion force can be identified as the equation shown below:

$$\\ \begin{matrix}Γ = \frac{4 γ ( CW )}{{{E}^{} ( CH )}^{2}} & ( 3 )\end{matrix}$$

where E is the Young's modulus; CW stands for channel width.

COMSOL simulation is also used in certain embodiments of the present invention to predict the collapsed structure and design the channel structure (FIGS. 3B and 3C). It was reported that such a collapse phenomenon is spontaneous and insensitive to the force applied to the elastomer, which is consistent with the observations in the present disclosure. This phenomenon also provides a convenient and reliable way of producing cost-efficient nanofiltration channels (FIG. 3D).

The second and more important hurdle is the flow resistance problem that a linear filtration structure faces. To make cells densely pack up from one end of the linear channel, a straightforward idea is to fabricate outlets only at the end of the filter structure. This was demonstrated to work for a shorter filter structure, but due to the size of bacteria cells, resistance to flow, and thereby pressure, will quickly build up before a visible length of the pile is generated in a longer filter structure. When considering whether to open nanopores on the sidewall of the channel, it was unclear whether cells would pile up at the end of the channel or on the pores on the sidewall; a previous report on mammalian cell filtration observed uniform packing of cells on all the pores of a square wave filter structure (FIG. 3A1). However, the accumulation behavior of cells in a linear filter can be affected by geometric factors such as the number and size of the pores, as well as the shape of the channel. In general, when the pores are relatively much smaller than the dimensions of the channel, most of the cells will not be in the local flow field towards the pore and will instead be carried downstream by the laminar flow in the channel. If a cell touches the accumulated pile around a pore, whether it will leave the pile depends on three forces: the drag of the net flow towards the pore, the drag of the net flow towards downstream, and the lift by the velocity gradient around the cell, as the flow velocity close to a solid surface is lower than that in bulk. The percentage flow rate into individual pores is inversely proportional to the number of pores; additionally, when a pore is blocked by cells, the density of net flow at the top of the pile into the pore will decrease with an increasing number of cells on the pile. It is therefore hypothesized that when there is a large number of nanopores on the sidewall of the channel, the effect of the latter two forces will have a chance to overwhelm the former force and the cell on a pile next to a pore will tend to go downstream. As a result, the accumulation of cells will start from the end of the channel after a negligible amount of cells have blocked the pores on the sidewall (FIG. 3A2). This will realize the cell-number ruler function desired for the barcode sensor.

Comparison of the Accumulating Behavior of the Bacteria within a Different Concentration of bacteria and operating flowrate

To verify the hypothesis described hereinbefore, fluid dynamics simulations using the fluid flow module of COMSOL are performed.

Three different concentrations of bacterial suspension [GFP-expressing E. coli] have been used to test the performance of the adaptive linear filter; the suspension was injected into the filter and the performance of the bacteria accumulation have been compared within an equal period (FIG. 25). The concentration of the bacterial suspension was evaluated by using a UV/Vis spectrophotometer, and the optical density for three different suspensions at 600 nm was OD 600 0.507 (˜4.05×10⁸ cells) (FIG. 25A), OD600 0.252 (˜2.01×10⁸ cells) (FIG. 25B) and OD600 0.0470(˜3.75×10⁷ cells) (FIG. 25C), respectively. Despite the fact that the concentrations of the bacterial suspensions were different, a linear relationship had been found for the accumulated bacteria within time under the same flow rate.

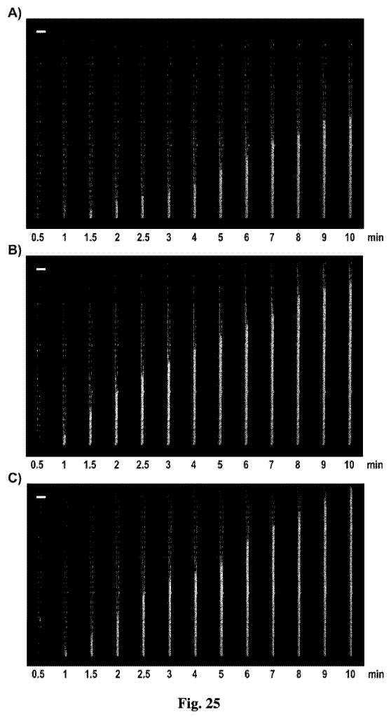

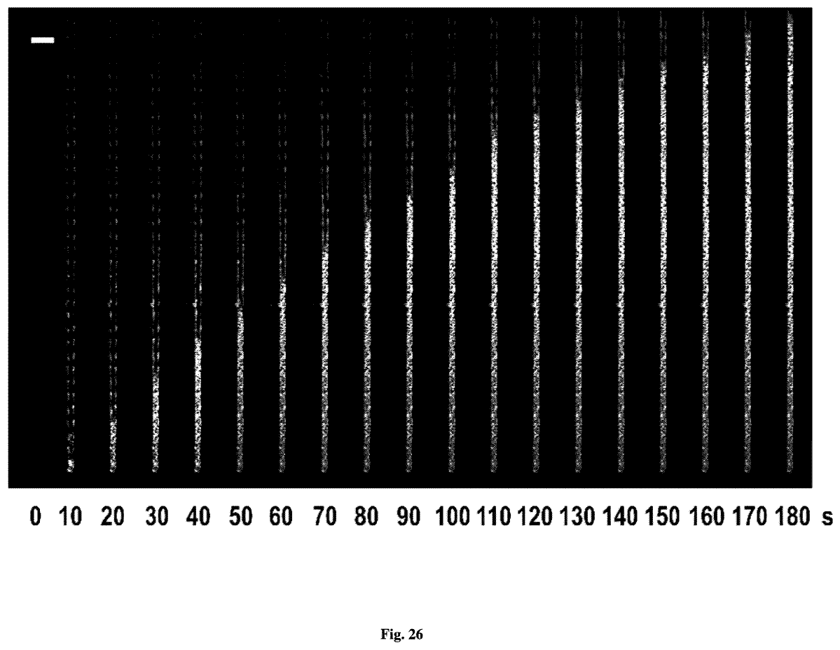

Except for the bacterial concentration, the flow rate is another decisive factor to affect the bacterial accumulative performance of the adaptive linear filter. In fact, the adhesion of bacteria to the polymer surface will depend on lots of different factors, such as the bacterial hydrophobicity and characteristics, the hydrophobicity of polymer's surface¹ etc. And according to different strengths of bacterial adherence, different flow rate (0.15 μL/min-1.5 μL/min) can be used to flush the bacteria inside the cultivation chamber out to the downstream “barcode” cell sensor for inspection. Under the invariant concentration of bacteria suspension, bacteria accumulate faster at a higher flow rate; the same length of accumulated bacteria can be achieved within 3 minutes at 2 μL/min (FIG. 26) compared to 10 minutes at 1 μL/min (FIG. 25). Nevertheless, partial debonding of the channelize channels may happen due to a high flow rate which may malfunction the adaptive linear filter should also be considered during the experimental design.

The simulation results show that with an increasing number of openings made on the sidewall of a channel, the flowrate near a cell-blocked pore decreases, and thus the ratio of bulk flow to the downstream decreases (FIG. 3C). When comparing the direction of net flow at different positions, the net flow in the channel is generally downstream, and it quickly turns into the sidewall pores at the end of the channel, indicating that cells suspended in the bulk flow will migrate to the end of the channel and pile up there. So, although there are nanopores on the sidewall of the channel, bacterial cells do not keep piling up from the sidewall, but instead accumulate from the end of the channel, thereby being called as an adaptive linear filter.

Based on the findings in the calculations and simulations, the devices (FIG. 23) are fabricated and tested using E. coli and S. aureus. To effectively filter bacteria, the apertures of the side nanochannels are made with an inner circle of ˜0.5 μm (FIG. 3C). When a bacterial suspension was injected into the filter channel, the cells compressed into a bar starting from the end of the channel; after crystal violet staining, images of the formed bars were taken. To understand the cell accumulation behavior of the barcode cell sensor, S. aureus were used as a model for calculation, which is a spherical shaped bacterium with a diameter of around 0.85 μm. To be detectable by a cellphone with a macro lens adaptor, the minimum length of the microbar was found to be about 0.025 mm. In order to fill the channel for a length of 0.025 mm, about 1.00×10⁴ cells are needed, based upon a calculation using the model described herein above. According to the measurement in the present disclosure, around 1.04×10⁴ cells are required to fill every 0.025 mm, which is in line with the initial expectation and proves the present sensor's reliability. The present barcode-like cell sensor has shown high linearity against the accumulation of bacteria cells under different cell number conditions and flow rates (FIG. 3E, FIG. 25A-C and FIG. 26), illustrating the reliability of the sensor.

### Rapid AST Using “Barcode” Cell Sensor

AST is used to determine the effectiveness of an antibiotic against a sample bacterium sample. In AST, the lowest concentration of antibiotic that will inhibit bacterial proliferation is measured, commonly known as the MIC value. To obtain the MIC value, the sample must be treated with multiple concentrations of the same antibiotic, which is usually labor intensive and time-consuming. The microfluidic gradient generator in the culture zone enables a low-cost solution to automatically generate the different concentrations; this means adding more levels of antibiotic concentration does not increase the labor cost. However, the microfluidic method still requires use of microscopes or other complicated equipment for MIC value determination. The formation of barcodes using the linear filter array, on the other hand, enables observation of the rapid response of cells to an AST without the requirement of a microscope. A mobile application for cell phones is provided to automatically record and analyze the testing results (FIGS. 22A, 22B and 22C1-C3). This not only simplifies the operation, but also reduce the amount of training needed for users, which is helpful for on-site implementation.

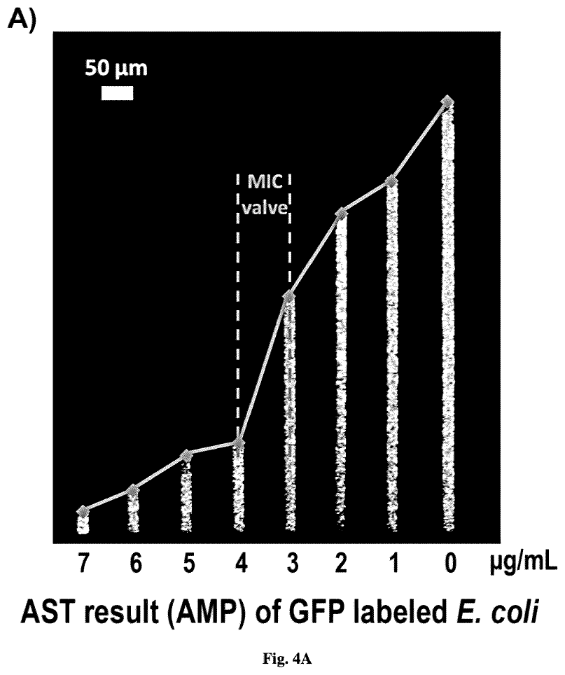

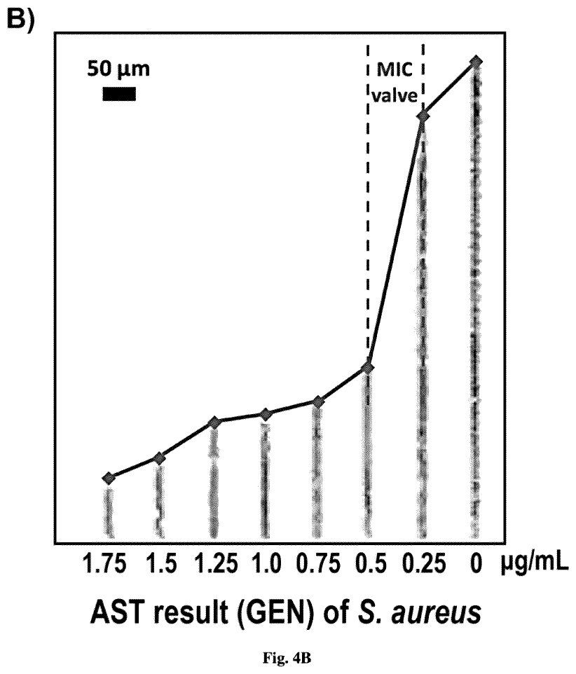

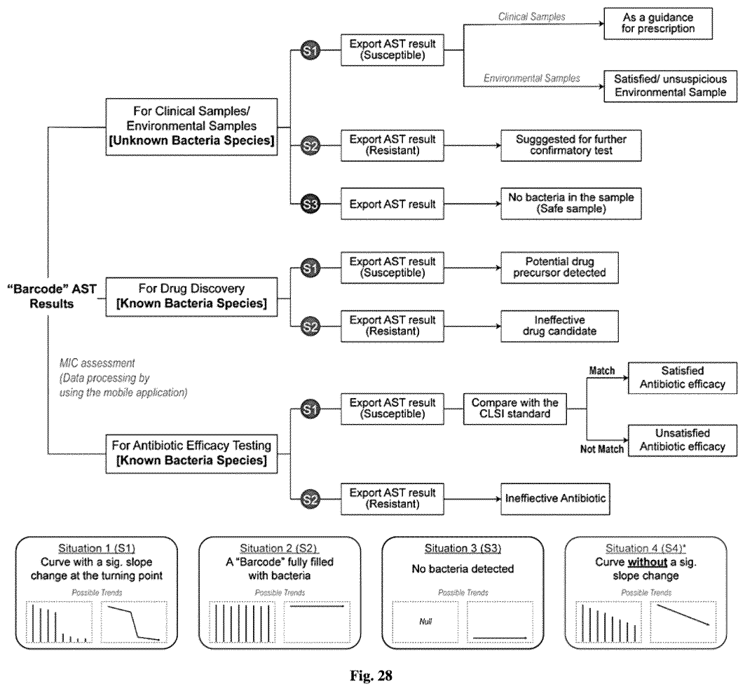

For evaluating the performance of the present barcode AST system, the ASTs are conducted for different bacteria using different antibiotics. FIGS. 4A and 4B show the AST results of GFP-labeled E. coli against ampicillin, and S. aureus against gentamicin after gram staining, respectively. In order to determine the MIC values using the present barcode cell sensor, the length of each accumulated bacteria microbar is converted into a value of length by the custom-developed application on a cell phone. The bar length values from different drug concentrations are then converted into a curve (FIG. 4) and the point with the most significant slope is determined as the MIC value (FIG. 21). In the following two cases, however, an MIC value is not generated. First, the lengths of all the bars are within the detection limit. Second, the length of the bar corresponding to the largest tested drug concentration is more than 50% of the length of the bar with no drug exposure (FIG. 28). When these two cases appear, it is recommended to repeat the test to validate. If the result is confirmed, it will be regarded in the first case that no bacteria is detected from the sample under the testing conditions, and in the second case that the bacteria in the sample is resistant to the tested antibiotic. The AST results of different antibiotics obtained using the present barcode AST system are listed in Table 3. AST results assessed using the standard broth microdilution method and the provided reference ranges from CLSI are also listed for comparison, which prove the consistency of the present method with current standards. While in the previous section the sensitivity of the barcode sensor is discussed, it is worthy of noting that, similar to other culture-based detection methods, the sensitivity for a real sample is dependent on the total processing time. Thus, turnaround time of the analysis can be adjusted towards desired sensitivity. In general, the turnaround time is affected by three factors. The first factor is the sample pretreatment process, which usually involves methods such as filtration and centrifugation to remove non-bacteria particles from the sample. Compared to conventional ASTs, the present method can save the time and labor for preparing samples in multiple drug concentrations. The second factor is on-chip incubation time. A longer culture period will allow for more cell doubling cycles to occur, thereby increasing detection sensitivity. Compared to conventional methods, the present method does not need to wait for macroscopic changes in the sample through doubling, but rather exploits the linear filter array to concentrate a smaller number of cells into miniature bars, which saves a significant amount of time. A typical incubation time is 3 hours; this allows 23/n (n is the generation time of the bacteria in hours) difference in numbers between inhibited cells and normal cells when treated with different concentrations of antibiotics. For example, the generation time for the two strains used in current work, E. coli and S. aureus, are around 0.3 hours and 0.5 hours, respectively. Note that the generation cycles affect the limit of detection for the analysis. Taking S. aureus as an example, the 3-hours incubation allows 6 cycles of division, which will increase the cell number by 64 times. As mentioned above, 1.04×10⁴ cells are generally required for the minimum length of detectable bar, thus ˜0.5×10² cells are needed for the sample in each of the deepened culture chambers before the on-chip cell culture. The last factor is the time for data acquisition. The data acquisition process in conventional methods is either labor-consuming, skill dependent, or instrument reliant. The present system uses a cell phone to record the barcode results and automatically process the data, making the process rapid, convenient, and free of instrument or skill requirements. The time required for pushing the cultured cells from the deepened chambers to form the barcode pattern in the linear filters takes less than 15 minutes. Based on this measurement, a satisfactory recovery rate is maintained up to the flowrate of 1 μL/minute through each linear filter. Beyond this flow rate, the recovery rate may gradually decrease, probably because of the deformation of channel structure or compression of cells under high flow rate pressure. Counting all three factors together, a typical analysis with the present system can be completed within 2-3 hours.

TABLE 3
Comparison of the AST results among the present barcode cell sensor, other
AST methods (i.e., MIC-broth microdilution), and standards from CLSI.

| | | | MIC-Broth Microdilution | | | |
|---|---|---|---|---|---|---|
| | MIC-“barcode” Chip(μg/mL) | | (μg/mL) | | MIC-CLSI (μg/mL) | |
| | E. coli | S. aureus | E. coli | S. aureus | E. coli | S. aureus |
| Ampicillin | 3-4 | 1.5-2 | 2-4 | 1-2 | 2-8 | 0.5-2 |
| Gentamicin | 0.25-0.5 | 0.25-0.5 | 0.25-0.5 | 0.25-0.5 | 0.25-1 | 0.12-1 |
| Tetracycline | 1.5-2 | 0.2-0.4 | 1-2 | 0.2-0.4 | 0.5-2 | 0.12-1 |
| Erythromycin | Resistant | 0.4-0.6 | Resistant | 0.25-1 | Resistant | 0.25-1 |

### Real Sample Analysis

To demonstrate the present AST system and evaluate its utility and reliability, real environmental samples were analyzed. Different samples were collected from public water dispensers in various venues. In order to access the hygiene level of a public water dispenser, a sterile swab was used to wipe the nozzle (about 4 cm2) of the water dispenser; subsequently, the swab was rinsed and the rinsing solution was used as the sample. After simple pre-incubation and enrichment of the bacteria in the sample suspension off chip, AST was performed using the present system (FIGS. 5A-5C). For a more complicated sample, pre-incubation inside a selective growth medium, followed by dilution plating, is suggested.

To better understand the types of bacterial species that generally appear on the water dispenser, the bacterial identification through Matrix-Assisted Laser Desorption Ionization Time of Flight Imaging Mass Spectrometry (MALDI-TOF IMS) was performed after the general bacterial isolation process (FIGS. 6A-6C).

Based on the findings in the present disclosure, multiple bacterial species have been recognized in the testing samples, including bacterial species that have the potential to form biofilm and colonize on the surface of the water dispenser (Table 2). Traditional broth microdilution AST was also performed to verify the AMR assessment results of the present sensor according to the approved standard from CLSI (FIGS. 27A and 27B, Table 4). Together, these results indicate that the current level of focus of scientists and the public on environmental-related AMR is still far from sufficient. As AMR microbes can exist in the environment all around us, close enough to come into contact with in daily life, they may pose a significant threat to the effective treatment of bacterial infections in the near future if no action is taken. Besides its enormous potential under the resource-limited condition and for the mass-screening ability that facilitates especially environmental-related AMR studies, the present AST system will also be compatible with the clinical AST, antibiotic efficacy testing, and drug discovery process (FIG. 28). Unlike standard clinical ASTs, the present barcode-like cell sensor can be used to provide a rapid and resource-independent AMR assessment for screening massive suspected samples at the same time. Preliminary information about the AMR assessments and MIC results of suspicious samples can then be obtained within 2 to 3 hours after sample collection. If situation 4 (FIG. 28) happens, performing a retest by increasing the concentration of antibiotics is suggested.

TABLE 4
AST results for the bacterial isolates obtained from the drinking water samples.

| Antimicrobial | | | | | | | | |
|---|---|---|---|---|---|---|---|---|
| Agents | Isolate 1 | Isolate 2 | Isolate 3 | Isolate 4 | Isolate 5 | Isolate 6 | Isolate 7 | Isolate 8 |
| Gentamicin | 4 | 16 | 16 | 16-32 | 4 | 2-4 | 16-32 | 16-32 |
| (μg/mL) | | | | | | | | |
| Ampicillin | R | R | R | R | R | R | R | R |
| (μg/mL) | | | | | | | | |

R stands for resistant.

### CONCLUSION

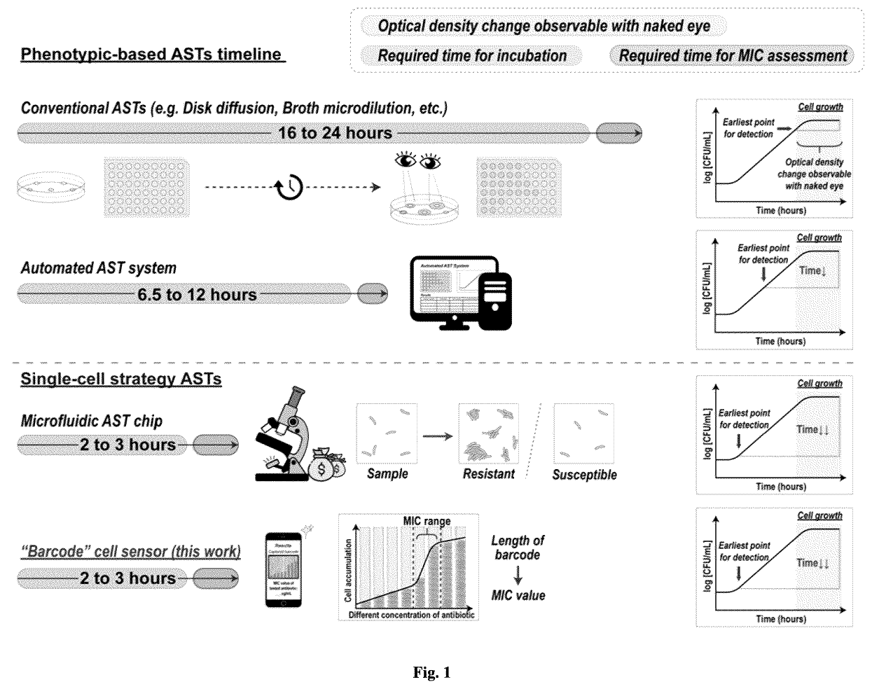

The present invention provides a novel, rapid, and easy to use platform for resource-independent, high throughput on-site AST. This platform can serve as a cost-efficient sample screening tool to quickly detect any potential drug-resistant bacteria, which can then be sent for subsequent advanced analysis. Besides the environmental AST provided in the present disclosure, this universal AST platform is also applicable for clinical AST at resource-limited conditions or when a massive screening is of urgent need, e.g., in the case of an outbreak or pandemic. This function is enabled by an adaptive linear filter of the present invention, which contains nanochannels at the side of a dead-end channel. When a bacterial suspension passes through this filter, the bacterium cells will accumulate in one end of the channel and form a miniature bar, the length of which is proportional to the number of cells in the suspension. When these filters are connected to the outlets of a microfluidic drug gradient, on-chip culture channel system, rapid AST results can be obtained by comparing the length of the formed bars, which form a barcode-like cell sensor. An application for smartphones is provided in the present disclosure to capture an image of the barcode and automatically analyze the testing results. The model strains and real-world sample tests showed reliable performance of the system; a typical test can be completed within 2 to 3 hours (FIG. 1). The present system is expected to become a useful tool for the routine screening of drug-resistant bacteria in different situations, such as the food industry, public areas, and healthcare facilities, which can be used without the need of advanced clinical assay facilities and operator skills.

### INDUSTRIAL APPLICATION

The present invention relates to a cell sensor for counting very small volumes of cells based on an adaptive linear filter array. More particularly, it relates to a method of using a fully automatic and microscope-free cell sensor incorporating the adaptive linear filter array (or interchangeably named as barcode cell sensor in the present disclosure), where suspended cells concentrate into microbars with various lengths proportional to the number of cells, thereby providing a quantitative analytical platform which result can be easily assessed by a portable device installed with a corresponding software program.

1. A microfluidic-based platform for implementing a one-pot antimicrobial susceptibility testing including cell culture, drug-cell incubation, and microscope-free quantitative analysis of viable cells after treatment by one or more compounds, the microfluidic-based platform comprises:
a thermal stage;
a light source; and
a visual inspection window,
the thermal stage comprising a cell culture zone and a cell sensing zone,
the cell culture zone comprising at least three fluid inlets and a drug concentration gradient generator downstream with respect to the at least three fluid inlets, the drug concentration gradient generator comprising a plurality of diverging fluid channels diverging fluids downstream to at least two of the fluid inlets, and a plurality of micro-chambers each with one or more deepened microwells for cell culture;
the cell sensing zone comprising a plurality of adaptive linear filter channels connecting to multiple fluid outlets of the cell culture zone via a one-piece connector, each of the adaptive linear filter channels being configured to receive cultivated cells from the cell culture zone and subsequently the received cultivated cells will accumulate at a downstream end of each of the adaptive linear filter channels,
the light source being disposed adjacent to or proximal to the downstream end of the adaptive linear filter channels for illuminating a wavelength of light towards the accumulated cultivated cells at the downstream end of each of the adaptive linear filter channels in order to visualize the accumulated cultivated cells through the visual inspection window disposed above an enclosure of the plurality of adaptive linear filter channels and image signal derived thereof being able to be directly captured by a portable device equipped with an imaging function.

2. The microfluidic-based platform of claim 1, wherein each of the adaptive linear filter channels comprises at least one main channel and at least two side channels disposed in a parallel orientation with the at least one main channel, and the at least one main channel communicating with the at least two side channels through a plurality of nano-scale channelization channels disposed in an orthogonal orientation with respect to both the at least one main channel and the at least two side channels such that the fluid containing cultivated cells flowing through the at least one main channel of the adaptive linear filter channel are directed to the at least two side channels through the plurality of nano-scale channelization channels based on filtration effect.

3. The microfluidic-based platform of claim 2, wherein the at least one main channel and the at least two side channels have an identical cross-sectional area with an aspect ratio of channel height to channel width of less than 1.

4. The microfluidic-based platform of claim 3, wherein the at least one main channel and the at least two side channels have an average channel height of about 8 μm.

5. The microfluidic-based platform of claim 3, wherein the at least one main channel and the at least two side channels have an average channel width of about 16 μm.

6. The microfluidic-based platform of claim 5, wherein the nano-scale channelization channels have the same channel width as that of the at least one main channel and the at least two side channels and an average channel height of about 800 nm.

7. The microfluidic-based platform of claim 1, wherein the cell sensing zone comprises at least eight adaptive linear filter channels to form an array of adaptive linear filter channels with various lengths of visible microbars corresponding to various quantities of cells accumulated at each of the adaptive linear filter channels after treatment with the one or more compounds at different concentrations according to different fluid channels of the diverging fluid channels of the drug concentration gradient generator at the cell culture zone.

8. The microfluidic-based platform of claim 7, wherein the length of the visible microbar is proportional to a proliferation rate of viable cells to be accumulated at the corresponding adaptive linear filter channel after the cells being treated with the one or more compounds at a specific concentration from one of the diverging fluid channels of the drug concentration gradient generator at the cell culture zone.

9. The microfluidic-based platform of claim 1, wherein a drug-containing fluid is loaded into one of the fluid inlets disposed upstream with respect to the drug concentration gradient generator while a pure fluid is loaded into another fluid inlet also disposed upstream with respect to the drug concentration gradient generator.

10. The microfluidic-based platform of claim 2, wherein the fluid containing cells is loaded into the fluid inlet disposed upstream with respect to the micro-chambers.

11. The microfluidic-based platform of claim 1, wherein the accumulated cells at the adaptive linear filter channels are gram stained such that under the illumination by the light source the image signal derived from the gram stained cells is directly captured by the portable device equipped with the imaging function, and wherein the wavelength of light illuminated by the light source is within a visible light range.

12. The microfluidic-based platform of claim 1, wherein at least the cell culture zone and the cell sensing zone of the thermal stage is made of one or more thermoplastic materials that are biocompatible whilst the cells cultivated at the cell culture zone do not adhere on interior surface of the microfluidic channels of the cell culture zone when a pure fluid is loaded into one of the fluid inlets to flush the cultivated cells towards the downstream direction with respect to the fluid outlets of the cell culture zone.

13. The microfluidic-based platform of claim 12, wherein the thermoplastic materials comprise polypropylene.

14. A system for screening or evaluating an antimicrobial activity of a potential drug candidate against one or more microbes, comprising an enclosure housing the microfluidic-based platform of claim 1, a fully automated fluid pump, a main circuit board and multiple control components for controlling loading of different fluids to the fluid inlets of the platform, a reagent compartment connecting to one or more fluid inlets of the microfluidic-based platform, a drug-cell incubation compartment with a thermostat for controlling temperature of the cell culture zone where the one or more microbes are incubated with different concentrations of the potential drug candidate, and a shadowless light panel for capturing images of the visualized accumulated cells at the cell sensing zone of the microfluidic-based platform from the visible inspection window by a macroscopic lens equipped to a portable device.

15. A method for screening or evaluating an antimicrobial activity of a potential drug candidate against one or more microbes of interest, the method comprising:
loading the potential drug candidate into the reagent compartment of the system according to claim 14;
using a corresponding software program installed in a paired workstation or portable device to control power on/off of and flowrate of fluid generated by the automated fluid pump of the system;
loading the one or more microbes of interest to the corresponding fluid inlet of the microfluidic-based platform;
treating the one or more microbes with the potential drug candidate at different concentrations in the cell culture zone of the microfluidic-based platform under a constant temperature controlled by the thermostat for a sufficient time duration in the drug-cell incubation compartment of the system;
flushing the cell culture of the one or more microbes after treating with different concentrations of the potential drug candidate from the cell culture zone via the one-piece connector to the cell sensing zone of the microfluidic-based platform by activating an antimicrobial susceptibility test function of the system through the software program of the paired workstation or portable device;
capturing images of visible bars derived from the accumulated cultivated cells of the one or more microbes at the adaptive linear filter array of the cell sensing zone by an imaging module paired with the workstation or the portable device under an illumination by the shadowless light panel of the system;
converting the captured images into image data for various lengths of the visible bars;
comparing the image data for various lengths of the visible bars with a referenced image data to determine the antimicrobial activity of the potential drug candidate against the one or more microbes of interest.

16. The method of claim 15, wherein said treating the one or more microbes with the potential drug candidate at different concentrations in the cell culture zone of the microfluidic-based platform under a constant temperature controlled by the thermostat is for about 1 to 3 hours.

17. The method of claim 15, wherein the one or more microbes comprise antimicrobial resistant microbes.

18. A method for evaluating antimicrobial resistance of a microbe against a compound, comprising:
loading the compound into the reagent compartment of the system according to claim 14;
using a corresponding software program installed in a paired workstation or portable device to control power on/off of and flowrate of fluid generated by the automated fluid pump of the system;
loading microbe to the corresponding fluid inlet of the microfluidic-based platform;
treating the microbe with the compound at different concentrations in the cell culture zone of the microfluidic-based platform under a constant temperature controlled by the thermostat for a sufficient time duration in the drug-cell incubation compartment of the system;
flushing the cell culture of the microbe after treating with different concentrations of the compound from the cell culture zone via the one-piece connector to the cell sensing zone of the microfluidic-based platform by activating an antimicrobial susceptibility test function of the system through the software program of the paired workstation or portable device;
capturing images of visible bars derived from the accumulated cultivated cells of the microbe at the adaptive linear filter array of the cell sensing zone by an imaging module paired with the workstation or the portable device under an illumination by the shadowless light panel of the system;
converting the captured images into image data for various lengths of the visible bars;
comparing the image data for various lengths of the visible bars with a referenced image data to determine the antimicrobial resistance of the microbe against the compound.

19. The method of claim 18, wherein said treating the microbe with the compound at different concentrations in the cell culture zone of the microfluidic-based platform under a constant temperature controlled by the thermostat is for about 1 to 3 hours.

20. The method of claim 18, wherein the compound is one or more antimicrobials comprising gentamicin, ampicillin, tetracycline, and erythromycin; the microbe comprises one or more strains of bacteria.

21. A method for evaluating antimicrobial resistant microbes in a fluid sample, comprising loading one or more known antimicrobials into the reagent compartment of the system according to claim 14;
using a corresponding software program installed in a paired workstation or portable device to control power on/off of and flowrate of fluid generated by the automated fluid pump of the system;
loading the fluid sample to the corresponding fluid inlet of the microfluidic-based platform;
treating the fluid sample with the one or more known compounds at different concentrations in the cell culture zone of the microfluidic-based platform under a constant temperature controlled by the thermostat for a sufficient time duration in the drug-cell incubation compartment of the system;
flushing the cell culture of microbes in the fluid sample after treating with different concentrations of the one or more known compounds from the cell culture zone via the one-piece connector to the cell sensing zone of the microfluidic-based platform by activating an antimicrobial susceptibility test function of the system through the software program of the paired workstation or portable device;
capturing images of visible bars derived from the accumulated cultivated cells of the microbes at the adaptive linear filter array of the cell sensing zone by an imaging module paired with the workstation or the portable device under an illumination by the shadowless light panel of the system;
converting the captured images into image data for various lengths of the visible bars;
comparing the image data for various lengths of the visible bars with a referenced image data to determine identity and antimicrobial resistance of the microbes in the fluid sample against the known compounds.

22. The method of claim 21, wherein the one or more known antimicrobials comprise gentamicin, ampicillin, tetracycline, and erythromycin; the microbes in the fluid sample comprise antimicrobial resistant microbes.

23. The method of claim 21, wherein said treating the fluid sample with the one or more known compounds at different concentrations in the cell culture zone of the microfluidic-based platform under a constant temperature controlled by the thermostat is for about 1 to 3 hours.
